(19) United States
(12) Patent Application Publication
(10) Pub. No.: US 2011/0110940 A1
(43) Pub. Date: May 12, 2011

(54) Methods for Enhancing the Efficacy of Vascular Disrupting Agents

(75) Inventors: Robert Kerbel, Toronto (CA); Yuval Shaked, Toronto (CA)

(73) Assignee: OXIGENE, INC., South San Francisco, CA (US)

(21) Appl. No.: 12/937,211

(22) PCT Filed: Apr. 15, 2009
(86) PCT No.: PCT/US09/40706

§ 371 Date: Dec. 21, 2010

Related U.S. Application Data
(60) Provisional application No. 61/045,266, filed on Apr. 15, 2008.

(51) Int. Cl. A61K 39/395; A61P 35/00

(52) U.S. Cl. 424/133.1; 424/172.1

(57) ABSTRACT

This invention relates to methods for treating, preventing and/or managing cancer in a subject including enhancing the efficacy of a Vascular Disrupting Agent (e.g., a combretastatin or derivative thereof) by administering to the subject an α4β1 integrin antagonist sequentially or simultaneously in combination with said Vascular Disrupting Agent.

### I. BACKGROUND OF THE INVENTION

The National Cancer Institute has estimated that in the United States alone, 1 in 3 people will be struck with cancer during their lifetime. Moreover approximately 50% to 60% of people contracting cancer will eventually succumb to the disease. The widespread occurrence of this disease underscores the need for improved anticancer regimens for the treatment of malignancy.

Due to the wide variety of cancers presently observed, numerous anticancer agents have been developed to destroy cancer within the body. These compounds are administered to cancer patients with the objective of destroying or otherwise inhibiting the growth of malignant cells while leaving normal, healthy cells undisturbed.

Anticancer agents have been classified based upon their mechanism of action. One promising new class of chemotherapeutic are referred to as a Vascular Disrupting Agents (VDAs) (or alternatively, Vascular Damaging Agents, Vascular Targeting Agents (VTAs) or Anti-vascular agents). The primary mechanism of action of VDAs is “vascular targeting”, in which the neovasculature of solid tumors is selectively disrupted, resulting in a transient decrease or complete shutdown of tumor blood flow that results in secondary tumor cell death due to hypoxia, acidosis, and/or nutrient deprivation (Dark et al., Cancer Res., 57: 1829-34, (1997); Chaplin et al., Anticancer Res., 19: 189-96, (1999); Hill et al., Anticancer Res., 22(3):1453-8 (2002); Holwell et al., Anticancer Res., 22(2A):707-11, (2002). Vascular disrupting agents (VDAs) cause acute shutdown of the established tumor vasculature, which is followed by massive intratumoral hypoxia and necrosis.

While VDAs have strong activity against a variety of tumors, a viable rim of tumor tissue typically surrounds a massive necrotic tumor center after treatment. Rapid tumor regrowth can resume from this residual viable rim, driven by an acute systemic mobilization of bone marrow derived circulating endothelial precursor cells (CEPs) which home to the viable tumor rim and stimulate revascularization. Exposure of tumor-bearing mice to cytotoxiclike vascular disrupting agents (VDAs) can cause a very rapid (i.e. within hours) mobilization of bone marrow cells, some of which are CEPs, followed by their homing to the viable rim of tumor tissue (Shaked, et al. 2006. Science 313:1785-1787). This acute reactive host process contributes to the rapid regrowth of the drug treated tumors and thus compromises much of the initial antitumor effect induced by VDA treatment secondary to the tumor vascular occlusion and tumor hypoxia-inducing properties of such drugs (Tozer 2005; Tozer, et al. 1999. Cancer Res 59:1626-1634).

Thus an urgent need to provide methods for improving of VDA therapy by preventing tumor regrowth due to endothelial cell mobilization exists in the art. The inventors unexpectedly have discovered that CEP homing and retention in tumors can be blocked by an α4β1 integrin antagonist, thereby increasing VDA-induced tumoral necrosis.

### II. SUMMARY OF THE INVENTION

The present invention provides, in part, methods for producing an enhanced antitumor effect wherein a combination of agents is employed. In particular aspects, the methods of the invention comprise the administration (e.g., sequential administration or co-administration) of a Vascular Disrupting Agent (hereinafter, a “VDA”) and an α4β1 integrin antagonist (e.g., an anti-α4β1 integrin antibody). The methods of the present invention provide advantages such as greater overall therapeutic efficacy of VDA therapy, for example, by preventing tumor regrowth. Further, where a tumor to be treated is not optimally responsive (e.g. resistant) to treatment with a Vascular Disrupting Agent, use of the present combination therapy methods can nonetheless provide effective treatment.

In one aspect, the invention provides a method for producing an anti-tumor effect in an patient suffering from a cancer or tumor, the method comprising administering to the patient a VDA and an α4β1 integrin antagonist (e.g., an anti-α4β1 integrin antibody). The VDA may be administered at any time relative to administration of the α4β1 integrin antagonist. In one embodiment, the VDA and α4β1 integrin antagonist can be administered simultaneously to produce a potentiated antitumor effect. In another embodiment the VDA and α4β1 integrin antagonist can be administered sequentially in any order to produce a potentiated antitumor effect. In one preferred embodiment, an α4β1 integrin (e.g. an anti-α4β1 integrin antibody) is sequentially administered in any order with effective amounts of a VDA (e.g. a combretastatin). In a preferred embodiment, the anti-α4β1 integrin antibody, natalizumab, is sequentially administered in any order with an effective amount of a a combretastatin. In a still more preferred embodiment, combretastatin A-4 phosphate (CA4P) or combretastatin A-1 diphosphate (CA1P) is sequentially or simultaneously administered in any order with an effective amount of an anti-α4β1 integrin antibody.

In another aspect, the invention provides a pharmaceutical composition comprising a VDA (e.g., a combretastatin) and an α4β1 integrin antagonist. In a preferred embodiment, the α4β1 integrin antagonist is natalizumab.

In another aspect, the pharmaceutical composition can be present in a subtherapeutic dose for one or both individual agents, the agents (i.e., the VDA and α4β1 integrin antagonist) being more effective when used in combination. Alternatively, each agent can be provided at therapeutic doses for one or both individual agents, such as those found in the Physician's Desk Reference.

In another aspect, the present invention further provides pharmaceutical kits. Exemplary kits of the invention comprise a first pharmaceutical composition comprising an α4β1 integrin antagonist and a second pharmaceutical composition comprising a VDA (e.g., a combretastatin) together in a package. The α4β1 integrin antagonist and VDA can be present, for example, in a subtherapeutic dose for one or both individual agents, the agents being effective in combination and providing reduced side effects while maintaining efficacy. Alternatively, each agent can be provided at a therapeutic dose, such as those found for the agent in the Physician's Desk Reference.

In certain aspects, the present invention provides methods of administering a VDA, preferably a combretastatin or combretastatin derivative, together with an α4β1 integrin antagonist in order to potentiate the overall efficacy of the combination. In one embodiment, the VDA and α4β1 integrin antagonist are administered simultaneously. In other embodiments, the VDA and α4β1 integrin antagonist are administered sequentially. When administered sequentially, an α4β1 integrin antagonist can preferably be administered, for example, within 24 hours of the administration of the VDA, such as within 1-24 hours prior, 2-24 hours prior, 3-24 hours prior, 6-24 hours prior, 8-24 hours prior, or 12 to 24 hours prior to administration, or such as within 1-24 hours after, 2-24 hours after, 3-24 hours after, 6-24 hours after, 8-24 hours after, or 12 to 24 hours after administration of the VDA.

In other aspects, the invention provides a method for producing an anti-tumor effect in a subject suffering from cancer or a tumor, the method comprising administering to the patient a VDA and an α4β1 integrin antagonist in amounts effective therefor.

In another aspect, the invention provides a method for preventing tumor regrowth in a subject suffering from cancer or a tumor, the method comprising administering to the patient a VDA and an α4β1 integrin antagonist in amounts effective therefor.

In yet another aspect, the invention provides a method for inhibiting tumor-associated angiogenesis in a subject that is treated with a VDA, the method comprising administering to the patient an α4β1 integrin antagonist in amounts effective therefor.

In still another another aspect, the invention provides a method for inhibiting homing and retention of circulating endothelial progenitor (CEP) cells or other proangiogenic cells to the tumor of a subject that is treated with a VDA, the method comprising administering to the patient an α4β1 integrin antagonist in amounts effective therefor.

In certain embodiments of the invention, the α4β1 integrin antagonist is an antibody. In other embodiments, the chemokine antagonist is a small molecule.

In certain embodiments, the VDA is a combretastatin. In certain embodiments, the combretastatin is a combretastatin derivative of Formula V:

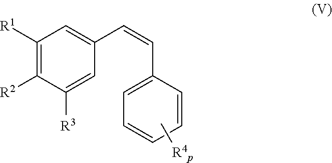

wherein
each of R¹, R² and R³, independently of the others, is selected from the group consisting of hydrogen, C1-6 alkoxy, and halogen, wherein at least two of R¹, R² and R³ are non-hydrogen;
R⁴ is selected from the group consisting of R⁵, R⁶, R⁵ substituted with one or more of the same or different R⁷ or R⁶, —OR⁷ substituted with one or more of the same or R⁷ or R⁶, —B(OR⁷)₂, —B(NR⁸R⁸)₂, —(CH₂)ₘ—R⁶, —(CHR⁷)ₘ—R⁶, —O—(CH₂)ₘ—R⁶, —S—(CH₂)ₘ—R⁶, —O—CHR⁷R⁶, —O—CR⁷(R⁶)₂, —O—(CHR⁷)ₘ—R⁶, —O—(CH₂)ₘ—CH[CH₂)ₘR⁶]R⁶, —S—(CHR⁷)ₘ—R⁶, —C(O)NH—(CH₂)ₘ—R⁶, —C(O)NH—(CHR⁷)ₘ—R⁶, —O—(CH₂)ₘ—C(O)NH—(CH₂)ₘ—R⁶, —S—(CH₂)ₘ—C(O)NH—(CH₂)ₘ—R⁶, —O—(CHR⁷)ₘ—C(O)NH—(CHR⁷)ₘ—R⁶, —S—(CHR⁷)ₘ—C(O)NH—(CHR⁷)ₘ—R⁶, —NH—(CH₂)ₘ—R⁶, —NH—(CHR⁷)ₘ—R⁶, —NH[(CH₂)ₘR⁶], —N[(CH₂)ₘR⁶]₂, —NH—C(O)—NH—(CH₂)ₘ—R⁶, —NH—C(O)—(CH₂)ₘ—CHR⁶R⁶ and —NH—(CH₂)ₘ—C(O)—NH—(CH₂)ₘ—R⁶;
each R⁵ is independently selected from the group consisting of C₁₋₆ alkyl, C₃₋₈ cycloalkyl, C₄₋₁₁ cycloalkylalkyl, C₅₋₁₀ aryl, C₆₋₁₆ arylalkyl, 2-6 membered heteroalkyl, 3-8 membered cycloheteroalkyl, 4-11 membered cycloheteroalkylalkyl, 5-10 membered heteroaryl, 6-16 membered heteroarylalkyl, phosphate, phosphate ester, phosphonate, phosphorodiamidate, phosphoramidate monoester, phosphoramidate diester, cyclic phosphoramidate, cyclic phosphorodiamidate, and phosphonamidate;
each R⁶ is a suitable group independently selected from the group consisting of ═O, —OR⁷, C1-3 haloalkyloxy, —OCF₃, ═S, —SR⁷, ═NR⁷, ═NOR⁷, —NR⁸R⁸, halogen, —CF₃, —CN, —NC, —OCN, —SCN, —NO, —NO₂, ═N₂, —N₃, —S(O)R⁷, —S(O)₂R⁷, —S(O)₂OR⁷, —S(O)NR⁸R⁸, —S(O)₂NR⁸R⁸, —OS(O)R⁷, —OS(O)₂R⁷, —OS(O)₂OR⁷, —OS(O)₂NR⁸R⁸, —C(O)R⁷, —C(O)OR⁷, —C(O)NR⁸R⁸, —C(NH)NR⁸R⁸, —C(NR⁷)NR⁸R⁸, —C(NOH)R⁷, —C(NOH)NR⁸R⁸, —OC(O)R⁷, —OC(O)OR⁷, —OC(O)NR⁸R⁸, —OC(NH)NR⁸R⁸, —OC(NR⁷)NR⁸R⁸, —[NHC(O)]ₙR⁷, —[NR⁷C(O)]ₙR⁷, —[NHC(O)]ₙOR⁷, —[NR⁷C(O)]ₙOR⁷, —[NHC(O)]ₙNR⁸R⁸, —[NR⁷C(O)]ₙNR⁸R⁸, —[NHC(NH)]ₙNR⁸R⁸ and -[NR⁷C(NR⁷)]ₙNR⁸R⁸;
each R⁷ is independently selected from the group consisting of hydrogen, C₁₋₆ alkyl, C₃₋₈ cycloalkyl, C₄₋₁₁ cycloalkylalkyl, C₅₋₁₀ aryl, C₆₋₁₆ arylalkyl, 2-6 membered heteroalkyl, 3-8 membered cycloheteroalkyl, 4-11 membered cycloheteroalkylalkyl, 5-10 membered heteroaryl, 6-16 membered heteroarylalkyl, phosphate, phosphate ester, phosphonate, phosphorodiamidate, phosphoramidate monoester, phosphoramidate diester, cyclic phosphoramidate, cyclic phosphorodiamidate, and phosphonamidate;
each R⁸ is independently R⁷ or, alternatively, two R⁸ are taken together with the nitrogen atom to which they are bonded to form a 5 to 8-membered cycloheteroalkyl or heteroaryl which may optionally include one or more of the same or different additional heteroatoms and which may optionally be substituted with one or more of the same or different R⁷ or suitable R⁶ groups;
each m independently is an integer from 1 to 3;
each n independently is an integer from 0 to 3;
p is an integer from 1 to 5, and
   wherein two adjacent R⁴ groups and their intervening atoms are bonded to form a 5-8 membered ring fused to the central phenyl group.

In a particularly preferred embodiment, the combretastatin agent is a compound of Formula II:

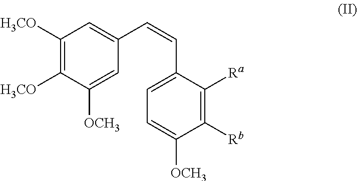

or a pharmaceutically acceptable salt thereof wherein
R^{a} is H, phosphate, phosphate ester, phosphonate, phosphoramidate monoester, phosphoramidate diester, cyclic phosphoramidate, phosphordiamidate, cyclic phosphorodiamidate, phosphonamidate or amino acid acyl; and
R^{b} is phosphate, phosphate ester, phosphonate, phosphoramidate monoester, phosphoramidate diester, cyclic phosphoramidate, phosphordiamidate, cyclic phosphorodiamidate, phosphonamidate or amino acid acyl.

In a preferred embodiment R^{a} is a phosphate of formula:

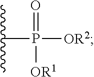

and
R^{b} is a phosphate of formula:

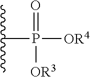

wherein OR¹, OR², OR³ and OR⁴ are each, independently, H, —O-QH+ or —O-M+,
wherein M+ is a monovalent or divalent metal cation, and Q is, independently:
a) an amino acid containing at least two nitrogen atoms where one of the nitrogen atoms, together with a proton, forms a quaternary ammonium cation QH+; or
b) an organic amine containing at least one nitrogen atom which, together with a proton, forms a quaternary ammonium cation, QH+.

In one embodiment, the combretastatin agent is a compound of Formula IIb:

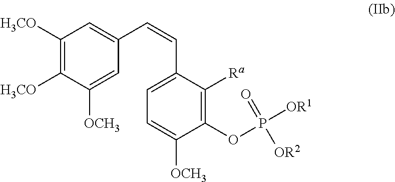

wherein Ra is H or OP(O)(OR³)OR⁴; and

OR¹, OR², OR³ and OR⁴ are each, independently, H, —O-QH+ or —O-M+,

wherein M+ is a monovalent or divalent metal cation, and Q is, independently:

a) an amino acid containing at least two nitrogen atoms where one of the nitrogen atoms, together with a proton, forms a quaternary ammonium cation QH+; or

b) an organic amine containing at least one nitrogen atom which, together with a proton, forms a quaternary ammonium cation, QH+.

In one embodiment, for Formula IIb, R³ is H or OP(O)(OR³)OR⁴, and R¹, R², R³ and R⁴ are each, independently, an aliphatic organic amine, alkali metals, transition metal, heteroarylene, heterocyclyl, nucleoside, nucleotide, alkaloid, amino sugar, amino nitrile, or nitrogenous antibiotic. In another embodiment, for Formula IIb, R¹, R², R³ and R⁴ are each, independently, Na, TRIS, histidine, ethanolamine, diethanolamine, ethylenediamine, diethylamine, triethanolamine, glucamine, N-methylglucamine, ethylenediamine, 2-(4-imidazolyl)-ethylamine, choline, or hydrabamine. In another embodiment, Formula II is represented by a compound of Formula III:

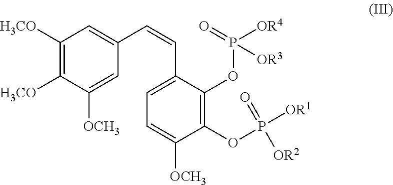

and pharmaceutically acceptable salts thereof.

In certain embodiments of the invention, the combretastatin agent is administered at a dose ranging from between 45 mg/kg and 63 mg/kg.

In certain embodiments of the invention, the cancer is selected from the group consisting of ovarian cancer, fallopian tube cancer, cervical cancer, breast cancer, lung cancer, melanoma, and primary cancer of the peritoneum. In other embodiments, the tumor is a solid tumor selected from the group consisting of a melanoma, an ovarian tumor, a cervical tumor, a breast tumor, small cell lung tumor, a non-small cell lung tumor, a fallopian tube tumor, and a primary tumor of the peritoneum.

In certain embodiments, the invention provides a method of treating a tumor in a subject in need thereof by administering to the subject a pharmaceutical composition comprising a compound of the Formula I and a compound of the Formula II or IIb wherein the compound of Formula I is administered first followed by administration of a compound of Formula II or IIb.

In other embodiments, the invention provides a method of treating a tumor in a subject in need thereof by administering to the subject a pharmaceutical composition comprising a compound of the Formula I and a compound of the Formula II or IIb, wherein the compound of Formula II or IIb is administered first followed by administration of a compound of Formula I.

In yet other embodiments, the invention provides a method of treating a tumor in a subject in need thereof by administering to the subject a pharmaceutical composition comprising a compound of the Formula I and a compound of the Formula II or IIb, wherein the compound of Formula I and a compound of Formula II or IIb are administered simultaneously.

In one preferred embodiment, the invention provides a method of treating a tumor in a subject in need thereof by administering to the subject a pharmaceutical composition comprising a natalizumab and CA1P. In another preferred embodiment, the invention provides a method of treating a tumor in a subject in need thereof by administering to the subject a pharmaceutical composition comprising natalizumab and CA4P.

In certain embodiments, the subject is a mammal. In one embodiment, the mammal is a human.

### III. BRIEF DESCRIPTION OF THE DRAWINGS

FIG. 1 illustrates the effect of treatment with a combretastatin and/or integrin antagonist. FIG. 1A illustrates evaluation of necrosis. FIG. 1B illustrates incorporation of bone marrow cells to the tumor vasculature. FIG. 1C provides tumor volumes measured three days after treatment and normalized to untreated control tumors.

FIG. 2 illustrates necrosis in LLC tumors from FIG. 1A.

### IV. DETAILED DESCRIPTION OF THE INVENTION

The invention is based, on the surprising and unexpected discovery that α4β1 integrin antagonist can prevent the regrowth or relapse of tumor growth that may occur following treatment of a solid tumor with a VDA. In particular, the inventors have discovered that α4β1 integrin antagonists can interfere with the recruitment and/or retention of bone-marrow-derived circulating endothelial progenitor cells (“CEPs”) within the tumor that occurs following treatment of the solid tumor with VDA therapy. CEPs have been shown to be a major determinant in tumor angiogenesis following VDA therapy (Shaked et al., Science, (2006), 313: 1785-1787). For example, VDAs can cause an rapid and pronounced increase (e.g., more that 3-4 fold) in the levels of CEPs in peripheral blood. These and other bone marrow derived proangiogenic cells home to the viable tumor rim which remains after VDA therapy and incorporate into the newly formed blood vessels, thereby contributing to tumor angiogenesis (e.g., by secreting growth factors such as vascular endothelial growth factor (VEGF)) and promoting the tumor re-growth that sometimes occurs following VDA treatment. The process whereby CEP and other bone marrow derived cells home to solid tumors is regulated in part by the secretion of the angiogenic chemokine factors (e.g., Stromal Cell Derived Factor 1 (SDF-1)) from solid tumors shortly following treatment with a VDA. These factors are thought to attract CXCR+ cells (e.g., CXCR4+ cells), and other cells which express cognate chemokine receptors on their cell surfaces, to the tumor site and promote their incorporation and retention in the solid tumor vasculature.

The inventors have made the surprising discovery that blocking the activity of proangiogenic chemokine receptors with a chemokine receptor antagonist can prevent the mobilization and retention of CEPs in a solid tumor (i.e., non-hematopoietic cancers) following VDA therapy and thereby enhance the efficacy of the VDA therapy. This result was highly unexpected since chemokine receptor antagonists have been known to augment (instead of inhibit) the mobilization of CEPs into peripheral blood (see, e.g., Shepherd et al., Blood, (2006), 108(12):3662-3667). Based on these previous findings, one skilled in the art would have expected that combination of a chemokine receptor antagonist (or chemokine antagonist) with a VDA would have further increased the mobilization of CEPs to a tumor, thereby reducing the efficacy of the VDA therapy. In contrast, the inventors have shown that combination therapy results in precisely the opposite therapeutic outcome.

So that the invention can be more clearly understood, the following definitions are provided:

As used herein, the term “effective amount” of a compound or pharmaceutical composition refers to an amount sufficient to provide the desired anti-cancer effect or anti-tumor effect in an animal, preferably a human, suffering from cancer. Desired anti-tumor effects include, without limitation, the modulation of tumor growth (e.g. tumor growth delay), tumor size, or metastasis, the reduction of toxicity and side effects associated with a particular anti-cancer agent, the enhancement of tumor necrosis or hypoxia, the reduction of tumor angiogenesis, the reduction of tumor re-growth, reduced tumor retention of CEPs and other pro-angiogenic cells, the amelioration or minimization of the clinical impairment or symptoms of cancer, extending the survival of the subject beyond that which would otherwise be expected in the absence of such treatment, and the prevention of tumor growth in an animal lacking any tumor formation prior to administration, i.e., prophylactic administration.

As used herein, the terms “modulate”, “modulating” or “modulation” refer to changing the rate at which a particular process occurs, inhibiting a particular process, reversing a particular process, and/or preventing the initiation of a particular process. Accordingly, if the particular process is tumor growth or metastasis, the term “modulation” includes, without limitation, decreasing the rate at which tumor growth and/or metastasis occurs; inhibiting tumor growth and/or metastasis, including tumor re-growth following treatment with an anticancer agent; reversing tumor growth and/or metastasis (including tumor shrinkage and/or eradication) and/or preventing tumor growth and/or metastasis.

“Synergistic effect”, as used herein refers to a greater-than-additive anti-cancer effect which is produced by a combination of two drugs, and which exceeds that which would otherwise result from individual administration of either drug alone. One measure of synergy between two drugs is the combination index (CI) method of Chou and Talalay (see Chang et al., Cancer Res. 45: 2434-2439, (1985)) which is based on the median-effect principle. This method calculates the degree of synergy, additivity, or antagonism between two drugs at various levels of cytotoxicity. Where the CI value is less than 1, there is synergy between the two drugs. Where the CI value is 1, there is an additive effect, but no synergistic effect. CI values greater than 1 indicate antagonism. The smaller the CI value, the greater the synergistic effect. Another measurement of synergy is the fractional inhibitory concentration (FIC). This fractional value is determined by expressing the IC50 of a drug acting in combination, as a function of the IC50 of the drug acting alone. For two interacting drugs, the sum of the FIC value for each drug represents the measure of synergistic interaction. Where the FIC is less than 1, there is synergy between the two drugs. An FIC value of 1 indicates an additive effect. The smaller the FIC value, the greater the synergistic interaction.

The term “anticancer agent” as used herein denotes a chemical compound or electromagnetic radiation (especially, X-rays) which is capable of modulating tumor growth or metastasis. When referring to use of such an agent with a combretastatin compound, the term refers to an agent other than a combretastatin compound. Unless otherwise indicated, this term can include one, or more than one, such agents. Thus, the term “anticancer agent” encompasses the use of one or more chemical compounds and/or electromagnetic radiation in the present methods and compositions. Where more than one anticancer agent is employed, the relative time for administration of the combretastatin compound can, as desired, be selected to provide a time-dependent effective tumor concentration of one, or more than one, of the anticancer agents.

As used herein, the term “combretastatin agent” or “combretastatin” denotes at least one member of the combretastatin family of compounds, derivatives or analogs thereof, their prodrugs (preferably phosphate prodrugs) and derivatives thereof, and salts of these compounds. Combretastatins include those anti-cancer compounds isolated from the South African tree Combreturn caffrum, including without limitation, Combretastatins A-1, A-2, A-3, A-4, B-1, B-2, B-3, B-4, D-1, and D-2, and various prodrugs thereof, exemplified by Combretastatin A-4 phosphate (CA4P) compounds, Combretastatin A-1 diphosphate (CA1P) compounds and salts thereof (see for example Pettit et al, Can. J. Chem., (1982); Pettit et al., J. Org. Chem., 1985; Pettit et al., J. Nat. Prod., 1987; Lin et al., Biochemistry, (1989); Pettit et al., J. Med. Chem., 1995; Pettit et al., Anticancer Drug Design, (2000); Pettit et al., Anticancer Drug Design, 16(4-5): 185-93 (2001)). Other exemplary prodrugs of combrestatin agents include the cyclic phosph(oramid)ate prodrugs described in U.S. Pat. Nos. 7,205,404 and 7,303,739, which are incorporated by reference herein. Exemplary combretastatin derivatives retain cis-stilbene as a fundamental skeleton and exhibit tubulin polymerization inhibiting activity of 10 micromolar or less (e.g., 1 micromolar, 0.1 micromolar, 10 nanomolar, 1 nanomolar or less).

As used herein, the term combretastatin A-4 phosphate (“CA4P”) denotes as least one of combretastatin A-4 phosphate prodrugs, derivatives thereof, and salts of these compounds. As used herein, the term combretastatin A-I diphosphate (“CA1P”) compound denotes as least one of combretastatin A-I diphosphate prodrugs (e.g., CA1P), derivatives thereof, and salts of these compounds.

As used herein, the term “prodrug” refers to a precursor form of the drug which is metabolically converted in vivo to produce the active drug. Thus, for example, combretastatin phosphate prodrug salts administered to an animal in accordance with the present invention undergo metabolic activation and regenerate combretastatin A-4 or combretastatin A-I in vivo, e.g., following dissociation and exposure to endogenous non-specific phosphatases in the body. Preferred prodrugs of the present invention include the phosphate, phosphate ester, phosphoramidate, phosphoramidate ester, or amino acid acyl groups as defined herein. Exemplary phosphate esters include —OP(O)(O-alkyl)₂ or salts of the phosphate group, for example —OP(O)(O—NH₄+)₂. In preferred embodiments, a prodrug of the invention comprises a substitution of a phenolic moiety or amine moiety of the active drug with a phosphate, phosphoramidate, or amino acid acyl group. A wide variety of methods for the preparation of prodrugs are known to those skilled in the art (see, for example, Pettit and Lippert, Anti-Cancer Drug Design, (2000), 15, 203-216).

As explained above, the present invention is directed towards a pharmaceutical composition that modulates growth or metastasis of tumors, particularly solid tumors, using a pharmaceutical composition of the present invention, along with methods of modulating tumor growth or metastasis, for example, with a pharmaceutical composition of the present invention.

The term “subject” is intended to include mammals suffering from or afflicted with a tumor. Exemplary subjects include humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from a cancer.

As used herein, the terms “tumor”, “tumor growth” or “tumor tissue” can be used interchangeably, and refer to an abnormal growth of tissue resulting from uncontrolled progressive multiplication of cells and serving no physiological function.

In particularly preferred embodiments, the methods of the invention are used to treat solid tumors. As is well-known in the art, solid tumors are quite distinct from non-solid tumors, such as those found in hemopoietic-related cancers. A solid tumor can be malignant, e.g. tending to metastasize and being life threatening, or benign. Examples of solid tumors that can be treated or prevented according to a method of the present invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, gastric cancer, pancreatic cancer, breast cancer, ovarian cancer, fallopian tube cancer, primary carcinoma of the peritoneum, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, liver metastases, bile duct carcinoma, choriocarcinoma, seminoma, embryonal carcinoma, thyroid carcinoma such as anaplastic thyroid cancer, Wilms' tumor, cervical cancer, testicular tumor, lung carcinoma such as small cell lung carcinoma and non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

In other embodiments, the methods of the invention are used to treat non-solid tumors. Examples of non-solid tumors include leukemias, such as myeloid leukemias and lymphoid leukemias, myelomas, and lymphomas. Particular forms of non-solid tumors include acute myelitic leukemia (AML), acute lymphatic leukemia (ALL), multiple myeloma (MM), chronic myelogenous leukemia (CML), hairy cell leukemia (HCL), acute promyelocytic leukemia (APL), and chronic lymphocytic leukemia (CLL). In a particularly preferred embodiment, the methods of the invention are used to treat chronic myelomonocytic leukemia (CMML).

In other embodiments, tumors comprising dysproliferative changes (such as metaplasias and dysplasias) can be treated or prevented with a pharmaceutical composition or method of the present invention in epithelial tissues such as those in the cervix, esophagus, and lung. Thus, the present invention provides for treatment of conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W.B. Saunders Co., Philadelphia, pp. 68 to 79). Hyperplasia is a form of controlled cell proliferation involving an increase in cell number in a tissue or organ, without significant alteration in structure or function. For example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder. For a review of such disorders, see Fishman et al., 1985, Medicine, 2d Ed., J. B. Lippincott Co., Philadelphia.

Other examples of tumors that are benign and can be treated or prevented in accordance with a method of the present invention include arteriovenous (AV) malformations, particularly in intracranial sites and myelomas.

The term “time-dependent effective tumor concentration,” as used herein, denotes a concentration of the other anticancer agent in the tumor tissue over time (i.e., from administration until the agent is cleared from the body) which potentiates the action of the combination of the combretastatin compound and other anticancer agent.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Alkylene” refers to divalent saturated aliphatic hydrocarbyl groups preferably having from 1 to 6 and more preferably 1 to 3 carbon atoms that are either straight-chained or branched. This term is exemplified by groups such as methylene (—CH₂—), ethylene (—CH₂CH₂—), n-propylene (—CH₂CH₂CH₂—), iso-propylene (—CH₂CH(CH₃)—) or (—CH(CH₃)CH₂—), and the like.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like.

“Acyl” refers to the groups H—C(O)—, alkyl-C(O)—, substituted alkyl C(O), alkenyl C(O), substituted alkenyl C(O), alkynyl C(O), substituted alkynyl C(O) cycloalkyl C(O), substituted cycloalkyl C(O), cycloalkenyl C(O), substituted cycloalkenyl C(O), aryl C(O), substituted aryl C(O), heteroaryl C(O), substituted heteroaryl C(O), heterocyclic C(O), and substituted heterocyclic C(O), wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH3C(O)—.

“Acylamino” refers to the groups —NR²⁰C(O)alkyl, —NR²⁰C(O)substituted alkyl, NR²⁰C(O)cycloalkyl, —NR²⁰C(O)substituted cycloalkyl, NR²⁰C(O)cycloalkenyl, NR²⁰C(O)substituted cycloalkenyl, —NR²⁰C(O)alkenyl, NR²⁰C(O)substituted alkenyl, NR²⁰C(O)alkynyl, —NR²⁰C(O)substituted alkynyl, NR²⁰C(O)aryl, NR²⁰C(O)substituted aryl, NR²⁰C(O)heteroaryl, NR²⁰C(O)substituted heteroaryl, NR²⁰C(O)heterocyclic, and NR²⁰C(O)substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Acyloxy” refers to the groups alkyl C(O)O, substituted alkyl C(O)O, alkenyl C(O)O, substituted alkenyl C(O)O, alkynyl C(O)O, substituted alkynyl C(O)O, aryl C(O)O, substituted aryl C(O)O, cycloalkyl C(O)O, substituted cycloalkyl C(O)O, cycloalkenyl C(O)O, substituted cycloalkenyl C(O)O, heteroaryl C(O)O, substituted heteroaryl C(O)O, heterocyclic C(O)O, and substituted heterocyclic C(O)O, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Aminocarbonyl” refers to the group C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonyl” refers to the group C(S)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group NR²⁰C(O)NR²¹R²², wherein R²⁰ is hydrogen or alkyl and R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminothiocarbonylamino” refers to the group NR²⁰C(S)NR²¹R²², wherein R²⁰ is hydrogen or alkyl and R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminocarbonyloxy” refers to the group —O—C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminosulfonyl” refers to the group —SO₂NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic and where R²¹ and R²² are optionally joined together with the nitrogen bound thereto to form a heterocyclic or substituted heterocyclic group and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminosulfonyloxy” refers to the group —O—SO₂NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic; R²¹ and R²² are optionally joined together with the nitrogen bound thereto to form a heterocyclic or substituted heterocyclic group; and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonylamino” refers to the group —NR²⁰—SO₂NR²¹R²² wherein R²⁰ is hydrogen or alkyl and R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Sulfonylamino” refers to the group —NR²¹SO₂R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amidino” refers to the group —C(═NR³⁰)NR³¹R³², wherein R³¹ and R³² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R³¹ and R³² are optionally joined together with the nitrogen bound thereto to form a heterocyclic or substituted heterocyclic group. R³⁰ is selected from the group consisting of hydrogen, alkyl, substituted alkyl, alkenyl, substituted alkenyl, alkynyl, substituted alkynyl, cycloalkynyl, substituted cycloalkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, nitro, nitroso, hydroxy, alkoxy, cyano, —N═N—N-alkyl, —N═N—N-substituted alkyl, —N(alkyl)SO₂-alkyl, —N(alkyl)SO₂-substituted alkyl, —N═N═N-alkyl, —N═N═N— substituted alkyl, acyl, —SO₂-alkyl and —SO₂-substituted alkyl, wherein alkyl, substituted alkyl, alkenyl, substituted alkenyl, alkynyl, substituted alkynyl, cycloalkynyl, substituted cycloalkynyl, cycloalkyl, substituted cycloalkyl, cycloalkenyl, substituted cycloalkenyl, aryl, substituted aryl, heteroaryl, substituted heteroaryl, heterocyclic, substituted heterocyclic, nitro, nitroso, hydroxy, alkoxy, and cyano are as defined herein. One of R³¹ and R³² along with R³⁰ are optionally joined together with the nitrogens bound thereto and the intervening carbon of the guanidine group to form a cyclic amidine.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like), provided that the point of attachment is through an atom of the aromatic aryl group. Preferred aryl groups include phenyl and naphthyl.

“Aryloxy” refers to the group —O-aryl, wherein aryl is as defined herein, including, by way of example, phenoxy, naphthoxy, and the like.

“Arylthio” refers to the group —S-aryl, wherein aryl is as defined herein. In other embodiments, sulfur may be oxidized to —S(O)— or —SO₂— moieties. The sulfoxide may exist as one or more stereoisomers.

“Alkenyl” refers to straight chain or branched hydrocarbyl groups having from 2 to 6 carbon atoms and preferably 2 to 4 carbon atoms and having at least 1 and preferably from 1 to 2 sites of double bond unsaturation. Such groups are exemplified, for example, by vinyl, allyl, and but 3 en 1 yl. Included within this term are the cis and trans isomers or mixtures of these isomers.

“Alkynyl” refers to straight or branched monovalent hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (—C≡CH), and propargyl (CH2CδCH).

“Alkynyloxy” refers to the group —O-alkynyl, wherein alkynyl is as defined herein. Alkynyloxy includes, by way of example, ethynyloxy, propynyloxy, and the like.

“Carboxyl” or “carboxy” refers to —COOH or salts thereof.

“Carboxyl ester” or “carboxy ester” refers to the groups C(O)O alkyl, C(O)O substituted alkyl, C(O)O alkenyl, C(O)O substituted alkenyl, C(O)O alkynyl, C(O)O substituted alkynyl, C(O)O aryl, C(O)O substituted aryl, C(O)O cycloalkyl, C(O)O substituted cycloalkyl, C(O)O cycloalkenyl, C(O)O substituted cycloalkenyl, C(O)O heteroaryl, C(O)O substituted heteroaryl, C(O)O heterocyclic, and C(O)O substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)amino” refers to the groups —NR—C(O)O alkyl, —NR—C(O)O-substituted alkyl, —NR—C(O)O alkenyl, —NR—C(O)O substituted alkenyl, —NR C(O)O-alkynyl, —NR—C(O)O-substituted alkynyl, —NR—C(O)O-aryl, —NR—C(O)O-substituted aryl, —NR—C(O)O-cycloalkyl, —NR—C(O)O-substituted cycloalkyl, —NR—C(O)O cycloalkenyl, —NR—C(O)O-substituted cycloalkenyl, —NR—C(O)O-heteroaryl, —NR—C(O)O-substituted heteroaryl, —NR—C(O)O-heterocyclic, and —NR—C(O)O-substituted heterocyclic, wherein R is alkyl or hydrogen and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” or “carbonate” refers to the groups —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” or “nitrile” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like.

“Cycloalkenyl” refers to non-aromatic cyclic alkyl groups of from 3 to 10 carbon atoms having single or multiple rings and having at least one double bond and preferably from 1 to 2 double bonds.

“Cycloalkynyl” refers to non-aromatic cycloalkyl groups of from 5 to 10 carbon atoms having single or multiple rings and having at least one triple bond.

“Cycloalkylene” refers to divalent cycloalkyl groups, wherein cycloalkyl is as defined herein.

“Cycloalkoxy” refers to —O-cycloalkyl.

“Cycloalkylthio” refers to —S-cycloalkyl. In other embodiments, sulfur may be oxidized to —S(O)— or —SO2-moieties. The sulfoxide may exist as one or more stereoisomers.

“Cycloalkenyloxy” refers to —O-cycloalkenyl.

“Cycloalkenylthio” refers to —S-cycloalkenyl. In other embodiments, sulfur may be oxidized to sulfinyl or sulfonyl moieties. The sulfoxide may exist as one or more stereoisomers.

“Guanidino” refers to the group —NHC(═NH)NH₂.

“Substituted guanidino” refers to the group —NR³³C(═NR³³)N(R³³)₂, wherein each R³³ independently is selected from the group consisting of hydrogen, alkyl, substituted alkyl, aryl, substituted aryl, heteroaryl, substituted heteroaryl, heterocyclic, and substituted heterocyclic; two R groups attached to a common guanidino nitrogen atom are optionally joined together with the nitrogen bound thereto to form a heterocyclic or substituted heterocyclic group, provided that at least one R is not hydrogen; and said substituents are as defined herein. Two R³³ groups on distinct nitrogens are optionally joined together with the nitrogens bound thereto and the intervening carbon of the guanidine group to form a cyclic guanidine.

“Halo” or “halogen” refers to fluoro, chloro, bromo, and iodo and is preferably fluoro or chloro.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen, and sulfur within the ring. Such heteroaryl groups can have a single ring (e.g., pyridinyl or fury) or multiple condensed rings (e.g., indolizinyl or benzothienyl), wherein the condensed rings may or may not be aromatic and/or contain a heteroatom, provided that the point of attachment is through an atom of the aromatic heteroaryl group. In one embodiment, the nitrogen and/or sulfur ring atom(s) of the heteroaryl group are optionally oxidized to provide for the N-oxide (N→O), sulfinyl, or sulfonyl moieties. Preferred heteroaryls include pyridinyl, pyrrolyl, indolyl, thiophenyl, and furanyl.

“Heteroaryloxy” refers to —O-heteroaryl.

“Heteroarylthio” refers to the group —S-heteroaryl. In other embodiments, sulfur may be oxidized to —S(O)— or —SO2-moieties. The sulfoxide may exist as one or more stereoisomers.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 15 ring atoms, including 1 to 4 hetero atoms. These ring atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In one embodiment, the nitrogen and/or sulfur atom(s) of the heterocyclic group are optionally oxidized to provide for the N-oxide, —S(O)—, or —SO₂— moieties.

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

“Heterocyclyloxy” refers to the group —O-heterocycyl.

“Heterocyclylthio” refers to the group —S-heterocycyl. In other embodiments, sulfur may be oxidized to —S(O)— or —SO₂— moieties. The sulfoxide may exist as one or more stereoisomers.

“Nitro” refers to the group —NO₂.

“Nitroso” refers to the group —NO.

“Oxo” refers to the atom (═O).

“Sulfonyl” refers to the group SO₂-alkyl, SO₂-substituted alkyl, SO₂-alkenyl, SO₂-substituted alkenyl, SO₂-cycloalkyl, SO₂-substituted cylcoalkyl, SO₂-cycloalkenyl, SO₂-substituted cycloalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes groups such as methyl-SO₂—, phenyl-SO₂—, and 4-methylphenyl-SO₂—.

“Sulfonyloxy” refers to the group —OSO₂-alkyl, O SO₂-substituted alkyl, OSO₂-alkenyl, OSO₂-substituted alkenyl, OSO₂-cycloalkyl, OSO₂-substituted cylcoalkyl, OSO₂-cycloalkenyl, OSO₂-substituted cycloalkenyl, OSO₂-aryl, OSO₂-substituted aryl, OSO₂-heteroaryl, OSO₂-substituted heteroaryl, OSO₂-heterocyclic, and OSO₂ substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thioacyl” refers to the groups H—C(S)—, alkyl-C(S)—, substituted alkyl-C(S—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S), substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thiol” refers to the group —SH.

“Thioxo” refers to the atom (═S).

“Alkylthio” refers to the group —S-alkyl, wherein alkyl is as defined herein. In other embodiments, sulfur may be oxidized to —S(O)—. The sulfoxide may exist as one or more stereoisomers.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

The term “substituted,” when used to modify a specified group or radical, means that one or more hydrogen atoms of the specified group or radical are each, independently of one another, replaced with the same or different substituent groups as defined below.

Substituent groups for substituting for hydrogens on saturated carbon atoms in the specified group or radical are, unless otherwise specified, —R⁶⁰, halo, —O⁻M⁺, ═O, —OR⁷⁰, —SR⁷⁰, —S⁻M⁺, ═S, —NR⁸⁰R⁸⁰, ═NR⁷⁰, ═N—OR⁷⁰, trihalomethyl, —CF₃, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂₀R⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)O R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R^{80'}s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means e.g. that one of the counter ions for such divalent alkali earth ions can be an ionized form of a compound of the invention and the other a typical counter ion such as chloride, or two ionized compounds of the invention can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

Substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

Substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In a preferred embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups are limited to substituted aryl-(substituted aryl)-substituted aryl.

The term “electron-withdrawing group” “or electron-withdrawing atom” is recognized in the art, and denotes the tendency of a substituent to attract valence electrons from neighboring atoms, i.e., the substituent is electronegative with respect to neighboring atoms. A quantification of the level of electron-withdrawing capability is given by the Hammett sigma (Σ) constant. This well known constant is described in many references, for instance, J. March, Advanced Organic Chemistry, McGraw Hill Book Company, New York, (1977 edition) pp. 251-259. The Hammett constant values are generally negative for electron donating groups (Σ[P]=−0.66 for NH2) and positive for electron withdrawing groups (Σ[P]=0.78 for a nitro group), wherein Σ[P] indicates para substitution. Non-limiting examples of electron-withdrawing groups include nitro, acyl, formyl, sulfonyl, trifluoromethyl, cyano, chloride, carbonyl, thiocarbonyl, ester, imino, amido, carboxylic acid, sulfonic acid, sulfinic acid, sulfamic acid, phosphonic acid, boronic acid, sulfate ester, hydroxyl, mercapto, cyano, cyanate, thiocyanate, isocyanate, isothiocyanate, carbonate, nitrate and nitro groups and the like. Exemplary electron-withdrawing atoms include, but are not limited to, an oxygen atom, a nitrogen atom, a sulfur atom or a halogen atom, such as a fluorine, chlorine, bromine or iodine atom. It is to be understood that, unless otherwise indicated, reference herein to an acidic functional group also encompasses salts of that functional group in combination with a suitable cation. Non-limiting examples of electron donating groups include, but are not limited to, a primary amino, secondary amino, tertiary amino, hydroxy, alkoxy, aryloxy, alkyl, or combinations thereof.

The description of the disclosure herein should be construed in congruity with the laws and principals of chemical bonding. For example, it may be necessary to remove a hydrogen atom in order accommodate a substitutent at any given location. Furthermore, it is to be understood that definitions of the variables (i.e., “R groups”), as well as the bond locations of the generic formulae of the invention, will be consistent with the laws of chemical bonding known in the art. It is also to be understood that all of the compounds of the invention described above will further include bonds between adjacent atoms and/or hydrogens as required to satisfy the valence of each atom. That is, bonds and/or hydrogen atoms are added to provide the following number of total bonds to each of the following types of atoms: carbon: four bonds; nitrogen: three bonds; oxygen: two bonds; and sulfur: two-six bonds.

As used herein, the term “pharmaceutically acceptable salt” includes salts that are physiologically tolerated by a subject. Such salts are typically prepared from an inorganic and/or organic acid. Examples of suitable inorganic acids include, but are not limited to, hydrochloric, hydrobromic, hydroiodic, nitric, sulfuric, and phosphoric acid. Organic acids may be aliphatic, aromatic, carboxylic, and/or sulfonic acids. Suitable organic acids include, but are not limited to, formic, acetic, propionic, succinic, camphorsulfonic, citric, fumaric, gluconic, lactic, malic, mucic, tartaric, para-toluenesulfonic, glycolic, glucuronic, maleic, furoic, glutamic, benzoic, anthranilic, salicylic, phenylacetic, mandelic, pamoic, methanesulfonic, ethanesulfonic, pantothenic, benzenesulfonic (besylate), stearic, sulfanilic, alginic, galacturonic, and the like. Other pharmaceutically acceptable salts include alkali metal cations such as Na, K, Li; alkali earth metal salts such as Mg or Ca; or organic amine salts such as those disclosed in PCT International Application Nos. WO02/22626 or WO00/48606 and U.S. Pat. Nos. 6,855,702 and 6,670,344, which are incorporated herein by reference in their entireties. Particularly preferred salts include organic amine salts such tromethamine (TRIS) and amino acid salts such as histidine. Other exemplary salts which can be synthesized using the methods of the invention include those described in U.S. Pat. No. 7,018,987, which is incorporated by reference herein.

A. Vascular Disrupting Agents (VDAs)

Vascular Disrupting Agents (“VDAs”), also known as vascular damaging agents or vascular targeting agents, are a separate class of antivascular chemotherapeutics. In contrast to anti-angiogenic drugs, which disrupt the new microvessel formation of developing tumors, VDAs attack solid tumors by selectively targeting the established tumor vasculature and causing extensive shutdown of tumor blood flow. A single dose of a VDA can cause a rapid and selective shutdown of the tumor neovasculature within a period of minutes to hours, leading eventually to tumor necrosis by induction of hypoxia and nutrient depletion. This vascular-mediated cytotoxic mechanism of VDA action is quite divorced from that of anti-angiogenic agents, which inhibit the formation of new tumor vascularization rather than interfering with the existing tumor vasculature. Other agents have been known to disrupt tumor vasculature, but differ in that they also manifest substantial normal tissue toxicity at their maximum tolerated dose. In contrast, genuine VDAs retain their vascular shutdown activity at a fraction of their maximum tolerated dose. It is thought that tubulin-binding VDAs selectively destabilize the microtubule cytoskeleton of tumor endothelial cells, causing a profound alteration in the shape of the cell which ultimately leads to occlusion of the tumor blood vessel and shutdown of blood flow to the tumor (Kanthou et al., Blood, 2002; Cooney et al., Curr Oncol Rep. 2005 7(2):90-5; Chaplin et al., Curr Opin Investig Drugs, (2006), 7(6):522-8).

A particularly promising subclass of VDAs are the combretastatins. Derived from the South African tree Combreturn caffrum, combretastatins such as combretastatin A-4 (CA-4) were initially identified in the 1980's as potent inhibitors of tubulin polymerization. CA-4, and other combretastatins (e.g. combretastatin A-1 (CA-1)) have been shown to bind a site at or near the colchicine binding site on tubulin with high affinity. In vitro studies clearly demonstrated that combretastatins are potent cytotoxic agents against a diverse spectrum of tumor cell types in culture. CA4P and CA1P, respective phosphate prodrugs of CA-4 and CA-1, were subsequently developed to combat problems with aqueous insolubility (see U.S. Pat. Nos. 4,996,237; 5,409,953; and 5,569,786, each of which is incorporated herein by reference). Surprisingly, CA1P and CA4P have also been shown to cause a rapid and acute shutdown of the blood flow to tumor tissue that is separate and distinct from the anti-proliferative effects of the agents on tumor cells themselves. A number of studies have shown that combretastatins cause extensive shut-down of blood flow within the tumor microvasculature, leading to secondary tumor cell death (Dark et al., Cancer Res., 57: 1829-34, (1997); Chaplin et al., Anticancer Res., 19: 189-96, (1999); Hill et al., Anticancer Res., 22(3):1453-8 (2002); Holwell et al., Anticancer Res., 22(2A):707-11, (2002). Blood flow to normal tissues is generally far less affected by CA4P and CA1P than blood flow to tumors, although blood flow to some organs, such as spleen, skin, skeletal muscle and brain, can be inhibited (Tozer et al., Cancer Res., 59: 1626-34 (1999)).

In light of the novel, non-cytotoxic, mode of action of combretastatins, there is considerable interest in exploiting the novel “vascular targeting” of these agents for cancer treatment. Single agent efficacy has been reported for CA4P using a frequent dosing regimen. Another report suggested that large tumors can, in some cases, be more responsive to CA4P therapy than small tumors. However, many tumors harvested from animals treated with CA4P reveal central necrosis surrounded by a rim of viable cells (Dark et al., Cancer Res., 57: 1829-34, (1997); Chaplin et al., Anticancer Res., 19: 189-96, (1999)). This rim of surviving cells is most likely a consequence of the shared normal vessel circulation between the perimeter of tumours and neighbouring normal tissue. The addition of an α4β1 integrin antagonist according to the present invention inhibits tumor regrowth from this rim of viable cells.

Exemplary combretastatin salts contemplated for use in the methods of the invention are described in WO 99/35150; WO 01/81355; U.S. Pat. Nos. 6,670,344; 6,538,038; 5,569,786; 5,561,122; 5,409,953; 4,996,237 which are incorporated herein by reference in their entirety.

Exemplary combretastatin derivatives or analogs of combretastatins are described in Singh et al., J. Org. Chem., 1989; Cushman et al, J. Med. Chem., 1991; Getahun et al, J. Med. Chem., 1992; Andres et al, Bioorg. Med. Chem. Lett., 1993; Mannila, et al., Liebigs. Ann. Chem., 1993; Shirai et al., Bioorg. Med. Chem. Lett., 1994; Medarde et al., Bioorg. Med. Chem. Lett., 1995; Wood et al, Br. J. Cancer, 1995; Bedford et al., Bioorg. Med. Chem. Lett., 1996; Dorr et al., Invest. New Drugs, 1996; Jonnalagadda et al., Bioorg. Med. Chem. Lett., 1996; Shirai et al., Heterocycles, 1997; Aleksandrzak, et al., Anticancer Drugs, 1998; Chen et al., Biochem. Pharmacol., 1998; Ducki et al., Bioorg. Med. Chem. Lett., 1998; Hatanaka et al., Bioorg. Med. Chem. Lett., 1998; Medarde et al., Eur. J. Med. Chem., 1998; Medina et al., Bioorg. Med. Chem. Lett., 1998; Ohsumi et al., Bioorg. Med. Chem. Lett., 1998; Ohsumi et al., J. Med. Chem., 1998; Pettit, et al., J. Med. Chem., 1998; Shirai et al., Bioorg. Med. Chem. Lett., 1998; Banwell et al., Aust. J. Chem., 1999; Medarde et al., Bioorg. Med. Chem. Lett., 1999; Shan et al., PNAS, 1999; Combeau et al., Mol. Pharmacol., 2000; Pettit et al., J. Med. Chem., 2000; Pinney et al., Bioorg. Med. Chem. Lett., 2000; Flynn et al., Bioorg. Med. Chem. Lett., 2001; Gwaltney et al., Bioorg. Med. Chem. Lett., 2001; Lawrence et al., 2001; Nguyen-Hai et al., Bioorg. Med. Chem. Lett., 2001; Xia et al., J. Med. Chem., 2001; Tahir et al., Cancer Res., 2001; Wu-Wong et al., Cancer Res., 2001; Janik et al, Bioorg. Med. Chem. Lett., 2002; Kim et al., Bioorg Med Chem. Lett., 2002; Li et al., Bioorg. Med. Chem. Lett., 2002; Nam et al., Bioorg. Med. Chem. Lett., 2002; Wang et al., J. Med. Chem. 2002; Hsieh et al., Bioorg. Med. Chem. Lett., 2003; Hadimani et al., Bioorg. Med. Chem. Lett., 2003; Mu et al., J. Med. Chem. 2003; Nam et al., Curr. Med. Chem., 2003; Pettit et al, J. Med. Chem., 2003; Gaukroger et al., Org Biomol Chem. 2003; Bailly et al., J Med Chem. 2003; Sun et al., Anticancer Res. 2004; Sun et al., Bioorg Med Chem Lett. 2004; Liou et al., J Med Chem. 2004; Perez-Melero et al., Bioorg Med Chem Lett. 2004; Liou et al., J Med Chem. 2004; Mamane et al., Chemistry. 2004; De Martini et al, J Med Chem. 2004; Ducki et al, J Med Chem. 2005; Maya et al., J Med Chem. 2005; Medarde et al., J Enzyme Inhib Med Chem. 2004; Simoni et al, J Med Chem. 2005; Sanchez et al., Bioorg Med Chem. 2005; Vongvanich et al., Planta Med. 2005; Tron et al., J Med Chem. 2005; Borrel et al., Bioorg Med Chem. 2005; Hsieh et al., Curr Pharm Des. 2005; Lawrence et al, Curr Pharm Des. 2005; Hadfield et al., Eur J Med Chem. 2005; Pettit et al., J Med Chem. 2005; Coggioloa et al., Bioorg Med Chem Lett. 2005; Kaffy et al., Org Biomol Chem. 2005; Mateo et al, J Org Chem. 2005; LeBlanc et al., Bioorg Med Chem. 2005; Srivistava et al., Bioorg Med Chem. 2005; Nguyen et al., J Med Chem. 2005; Kong et al., Chem Biol. 2005; Li et al, Bioorg Med Chem Lett. 2005; Pettit et al, J Nat Prod. 2005; Nicholson et al, Anticancer Drugs. 2006; Monk et al., Bioorg Med Chem. 2006; De Martino et al., J Med Chem. 2006; Peifer et al., J Med Chem. 2006; Kaffy et al., Bioorg Med Chem. 2006; Banwell et al., Bioorg Med Chem. 2006; Dupeyre et al., Bioorg Med Chem. 2006 Simoni et al, J Med Chem. 2006; Tron et al., J Med Chem. 2006; Romagnoli et al, J Med Chem. 2006; Pandit et al., Bioorg Med Chem. 2006; Nakamura et al., Chem Med Chem. 2006; Pirali et al., J Med Chem. 2006; Bellina et al., Bioorg Med Chem Lett. 2006; Hu et al, J Med Chem. 2006; Chang et al., J Med Chem. 2006; Thomson et al., Mol Cancer Ther. 2006; Fortin et al., Bioorg Med Chem Lett., 2007; Duan et al., J Med Chem., 2007; Zhang et al., J Med Chem. 2007; Wu et al., Bioorg Med Chem Lett. 2007; Sun et al., Bioorg Med Chem Lett. 2007, WO 07/140,662; WO 07/059,118; WO 06/138427; WO 06/036743; WO 05/007635, WO 03/040077, WO 03/035008, WO 02/50007, WO 02/14329; WO 01/12579, WO 01/09103, WO 01/81288, WO 01/84929, WO 00/48590, WO 00/73264, WO 00/06556, WO 00/35865, WO 99/34788, WO 99/48495, WO 92/16486, U.S. Pat. Nos. 7,312,241; 7,223,747; 7,220,784; 7,135,502; 7,125,906; 7,105,695; 7,105,501; 7,087,627; 7,030,123; 7,078,552; 7,030,123; 7,018,987; 6,992,106; 6,919,324; 6,846,192, 6,855,702; 6,849,656; 6,794,384; 6,787,672, 6,777,578, 6,723,858, 6,720,323, 6,433,012, 6,423,753, 6,201,001, 6,150,407, 6,169,104, 5,731,353, 5,674,906, 5,430,062, 5,525,632, 4,996,237 and 4,940,726, each of which are incorporated herein by reference in their entirety.

In one exemplary embodiment, a combretastatin derivate is the amine or serinamide derivative of CA4, e.g. AVE8032 (Aventis Pharma, France). In another exemplary embodiment, a combretastatin derivative is ZD6126 (AstraZeneca, UK).

In particular embodiments, a combretastatin derivative is a compound of Formula V:

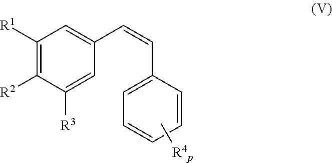

wherein
each of R¹, R² and R³, independently of the others, is selected from the group consisting of hydrogen, C1-6 alkoxy, and halogen, wherein at least two of R¹, R² and R³ are non-hydrogen;
R⁴ is selected from the group consisting of R⁵, R⁶, R⁵ substituted with one or more of the same or different R⁷ or R⁶, —OR⁷ substituted with one or more of the same or R⁷ or R⁶, —B(OR⁷)₂, —B(NR⁸R⁸)₂, —(CH₂)ₘ—R⁶, —(CHR⁷)ₘ—R⁶, —O—(CH₂)ₘ—R⁶, —S—(CH₂)ₘ—R⁶, —O—CHR⁷R⁶, —O—CR⁷(R⁶)₂, —O—(CHR⁷)ₘ—R⁶, —O— (CH₂)ₘ—CH[(CH₂)ₘR⁶]R⁶, —S—(CHR⁷)ₘ—R⁶, —C(O)NH—(CH₂)ₘ—R⁶, —C(O)NH—(CHR⁷)ₘ—R⁶,
—O—(CH₂)ₘ—C(O)NH—(CH₂)ₘ—R⁶, —S—(CH₂)ₘ—C(O)NH—(CH₂)ₘ—R⁶,
—O—(CHR⁷)ₘ—C(O)NH—(CHR⁷)ₘ—R⁶, —S—(CHR⁷)ₘ—C(O)NH—(CHR⁷)ₘ—R⁶, —NH—(CH₂)ₘ—R⁶,
—NH—(CHR⁷)ₘ—R⁶, —NH[(CH₂)ₘR⁶], —N[(CH₂)ₘR⁶]₂, —NH—C(O)—NH—(CH₂)ₘ—R⁶,
—NH—C(O)—(CH₂)ₘ—CHR⁶R⁶ and —NH—(CH₂)ₘ—C(O)—NH—(CH₂)ₘ—R⁶;
each R⁵ is independently selected from the group consisting of C₁₋₆ alkyl, C₃₋₈ cycloalkyl, C₄₋₁₁ cycloalkylalkyl, C₅₋₁₀ aryl, C₆₋₁₆ arylalkyl, 2-6 membered heteroalkyl, 3-8 membered cycloheteroalkyl, 4-11 membered cycloheteroalkylalkyl, 5-10 membered heteroaryl, 6-16 membered heteroarylalkyl, phosphate, phosphate ester, phosphonate, phosphorodiamidate, phosphoramidate monoester, phosphoramidate diester, cyclic phosphoramidate, cyclic phosphorodiamidate, and phosphonamidate each R⁶ is a suitable group independently selected from the group consisting of ═O, —OR⁷, C1-3 haloalkyloxy, —OCF₃, ═S, —SR⁷, ═NR⁷, ═NOR⁷, —NR⁸R⁸, halogen, —CF₃, —CN, —NC, —OCN, —SCN, —NO, —NO₂, ═N₂, —N₃, —S(O)R⁷, —S(O)₂R⁷, —S(O)₂OR⁷, —S(O)NR⁸R⁸, —S(O)₂NR⁸R⁸, —OS(O)R⁷, —OS(O)₂R⁷, —OS(O)₂OR⁷, —OS(O)₂NR⁸R⁸, —C(O)R⁷, —C(O)OR⁷, —C(O)NR⁸R⁸, —C(NH)NR⁸R⁸, —C(NR⁷)NR⁸R⁸, —C(NOH)R⁷, —C(NOH)NR⁸R⁸, —OC(O)R⁷, —OC(O)OR⁷, —OC(O)NR⁸R⁸, —OC(NH)NR⁸R⁸, —OC(NR⁷)NR⁸R⁸, —[NHC(O)]ₙR⁷, —[NR⁷C(O)]ₙR⁷, —[NHC(O)]ₙOR⁷, —[NR⁷C(O)]ₙ0R⁷, —[NHC(O)]ₙNR⁸R⁸, —[NR⁷C(O)]ₙNR⁸R⁸, —[NHC(NH)]ₙNR⁸R⁸ and -[NR⁷C(NR⁷)]ₙNR⁸R⁸; each R⁷ is independently selected from the group consisting of hydrogen, C₁₋₆ alkyl, C₃₋₈-cycloalkyl, C₄₋₁₁ cycloalkylalkyl, C₅₋₁₀ aryl, C₆₋₁₆ arylalkyl, 2-6 membered heteroalkyl, 3-8 membered cycloheteroalkyl, 4-11 membered cycloheteroalkylalkyl, 5-10 membered heteroaryl, 6-16 membered heteroarylalkyl, phosphate, phosphate ester, phosphonate, phosphorodiamidate, phosphoramidate monoester, phosphoramidate diester, cyclic phosphoramidate, cyclic phosphorodiamidate, and phosphonamidate;
each R⁸ is independently R⁷ or, alternatively, two R⁸ are taken together with the nitrogen atom to which they are bonded to form a 5 to 8-membered cycloheteroalkyl or heteroaryl which may optionally include one or more of the same or different additional heteroatoms and which may optionally be substituted with one or more of the same or different R⁷ or suitable R⁶ groups;
each m independently is an integer from 1 to 3;
each n independently is an integer from 0 to 3;
p is an integer from 1 to 5, and
wherein two adjacent R⁴ groups and their intervening atoms are bonded to form a 5-8 membered ring fused to the central phenyl group.

In a particularly preferred embodiment, the combretastatin agent is a compound of Formula II:

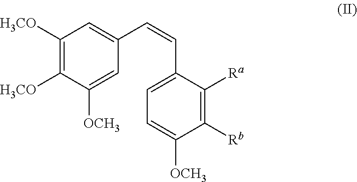

or a pharmaceutically acceptable salt thereof wherein R^{a} is H, phosphate, phosphate ester, phosphonate, phosphoramidate monoester, phosphoramidate diester, cyclic phosphoramidate, phosphordiamidate, cyclic phosphorodiamidate, phosphonamidate or amino acid acyl; and

R^{b} is phosphate, phosphate ester, phosphonate, phosphoramidate monoester, phosphoramidate diester, cyclic phosphoramidate, phosphordiamidate, cyclic phosphorodiamidate, phosphonamidate or amino acid acyl.

In a preferred embodiment R^{a} is a phosphate of formula:

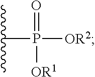

and R^{b} is a phosphate of formula:

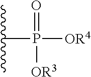

wherein OR¹, OR², OR³ and OR⁴ are each, independently, H, —O-QH+ or —O-M+,
wherein M+ is a monovalent or divalent metal cation, and Q is, independently:

a) an amino acid containing at least two nitrogen atoms where one of the nitrogen atoms, together with a proton, forms a quaternary ammonium cation QH+; or

b) an organic amine containing at least one nitrogen atom which, together with a proton, forms a quaternary ammonium cation, QH+.

In a particular embodiment, the combrestatin agent is a compound of the Formula IIb:

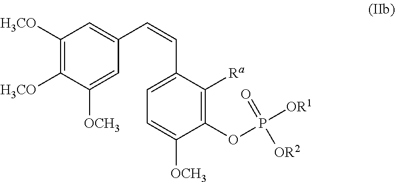

wherein

### R^{a} is H or OP(O)(OR³)OR⁴; and

OR¹, OR², OR³ and OR⁴ are each, independently, H, —O-QH+ or —O-M+,

wherein M+ is a monovalent or divalent metal cation, and Q is, independently:

a) an amino acid containing at least two nitrogen atoms where one of the nitrogen atoms, together with a proton, forms a quaternary ammonium cation QH+; or

b) an organic amine containing at least one nitrogen atom which, together with a proton, forms a quaternary ammonium cation, QH+.

In one embodiment of Formula IIb, R^{a} is H, one of OR¹ and OR² is hydroxyl, and the other is —O-QH+ where Q is L-histidine. In another embodiment of Formula IIb, R^{a} is H, one of OR¹ and OR² is hydroxyl and the other is —O-QH+ and Q is tris(hydroxymethyl)amino methane (“TRIS”).

In another embodiment of Formula IIb, R^{a} is H or OP(O)(OR³)OR⁴, and R¹, R², R³ and R⁴ are each, independently, an aliphatic organic amine, alkali metals, transition metals, heteroarylene, heterocyclyl, nucleoside, nucleotide, alkaloid, amino sugar, amino nitrile, or nitrogenous antibiotic.

In another embodiment of Formula IIb, R¹, R², R³ and R⁴ are each, independently, Na, TRIS, histidine, ethanolamine, diethanolamine, ethylenediamine, diethylamine, triethanolamine, glucamine, N-methylglucamine, ethylenediamine, 2-(4-imidazolyl)-ethylamine, choline, or hydrabamine.

In another embodiment, Formula IIb is represented by a compound of Formula III:

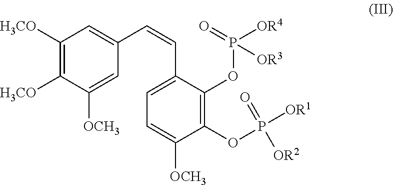

wherein OR¹, OR², OR³ and OR⁴ are each, independently, H, —O-QH+ or —O-M+, wherein M+ is a monovalent or divalent metal cation, and Q is, independently:

a) an amino acid containing at least two nitrogen atoms where one of the nitrogen atoms, together with a proton, forms a quaternary ammonium cation QH+; or

b) an organic containing at least one nitrogen atom which, together with a proton, forms a quaternary ammonium cation, QH+.

In one embodiment of Formula III, at least one of OR¹, OR², OR³ and OR⁴ is hydroxyl, and at least one of OR¹, OR², OR³ and OR⁴ is —O-QH+, where Q is L-histidine.

In another embodiment of Formula III, at least one of OR¹, OR², OR³ and OR⁴ is hydroxyl, and at least one of OR¹, OR², OR³ and OR⁴ is TRIS.

In another aspect, the invention provides a pharmaceutical composition comprising a compound of Formula I and a compound of Formula IV:

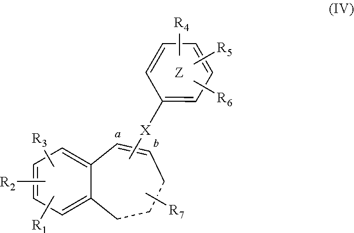

wherein
the dashed lines independently indicate a single or double bond;
X is selected from the group consisting of a single bond, CH₂, O, S, N(H), and C(O);

R¹, R², R³, R⁴, R⁵, R⁶ and R⁷ are each, independently, selected from the group consisting of H, halogen, lower alkyl, lower alkoxy, hydroxyl, amine, phosphate, phosphoramidate, and amino acid acyl group; and phenyl ring “Z” is bonded to either carbon “a” or “b.”

In one embodiment, the compound of Formula IV is selected from the group consisting of (1) 3-Methoxy-8-(3,4,5-trimethoxy-phenyl)-6,7-dihydro-5H-benzocycloheptenyl; (2) 3-Methoxy-9-(3,4,5-trimethoxy-phenyl)-6,7-dihydro-5H-benzocycloheptene; (3) 2-Methoxy-6-(3,4,5-trimethoxy-phenyl)-8,9-dihydro-7H-benzocyclohepten-1-ol; (4) 2-Methoxy-5-(3,4,5-trimethoxy-phenyl)-8,9-dihydro-7H-benzocyclohepten-1-ol; (5) 2-Methoxy-6-(3,4,5-trimethoxy-phenyl)-8,9-dihydro-7H-benzocyclohepten-1-ol; (6) 2-Methoxy-5-(3,4,5-trimethoxy-phenyl)-7,8,9,10-tetrahydro-benzocycloocten-1-ol; (9) 3-Methoxy-6-(1,2,3-trimethoxy-8,9-dihydro-7H-benzocyclohepten-5-yl)-benzene-1,2-diol; (11) 3-Methoxy-6-(1,2,3-trimethoxy-8,9-dihydro-7H-benzocyclohepten-5-yl)-benzene-1,2-diol; (12) 2-Methoxy-5-(1,2,3-trimethoxy-8,9-dihydro-7H-benzocyclohepten-5-yl)-phenol; (13) 3-Methoxy-6-(1,2,3-trimethoxy-7,8,9,10-tetrahydro-benzocycloocten-5-yl)-benzene-1,2-diol; (14) 2-Methoxy-5-(1,2,3-trimethoxy-7,8,9,10-tetrahydro-benzocycloocten-5-yl)-phenol; (31) 2-Methoxy-5-(1,2,3-trimethoxy-8,9-dihydro-7H-benzocyclohepten-6-yl)-phenol; (32) 3-Methoxy-6-(1,2,3-trimethoxy-8,9-dihydro-7H-benzocyclohepten-6-yl)-benzene-1,2-diol; (15) (1-Hydroxy-2-methoxy-8,9-dihydro-7H-benzocyclohepten-6-yl)-(3,4,5-trimethoxy-phenyl)-methanone; (16) (1-Hydroxy-2-methoxy-8,9-dihydro-7H-benzocyclohepten-5-yl)-(3,4,5-trimethoxy-phenyl)-methanone; (17) (1-Hydroxy-2-methoxy-7,8,9,10-tetrahydro-benzocycloocten-6-yl)-(3,4,5-trimethoxy-phenyl)-methanone; (18) (1-Hydroxy-2-methoxy-7,8,9,10-tetrahydro-benzocycloocten-5-yl)-(3,4,5-trimethoxy-phenyl)-methanone; (23) (2,3-Dihydroxy-4-methoxy-phenyl)-(1,2,3-trimethoxy-8,9-dihydro-7H-benzocyclohepten-5-yl)-methanone; (24) (3-Hydroxy-4-methoxy-phenyl)-(1,2,3-trimethoxy-8,9-dihydro-7H-benzocyclohepten-5-yl)-methanone; (25) (2,3-Dihydroxy-4-methoxy-phenyl)-(1,2,3-trimethoxy-7,8,9,10-tetrahydro-benzocycloocten-5-yl)-methanone and (26) (3-Hydroxy-4-methoxy-phenyl)-(1,2,3-trimethoxy-7,8,9,10-tetrahydro-benzocycloocten-5-yl)-methanone. Additional compounds of Formula IV are disclosed in International PCT publication No. WO 2006/138427A2, published Dec. 28, 2006, which is incorporated by reference herein in its entirety.

B. Integrin Antagonists

In certain aspects of the invention, a VDA is administered together with a integrin antagonist. The phrase “integrin antagonist” includes agents that impair endothelial cell adhesion via the various integrins. Integrins are a large family of cell surface glycoproteins which mediate cell adhesion and play central roles in many adhesion phenomena. Integrins are heterodimers composed of noncovalently linked alpha and beta polypeptide subunits. Currently eleven different alpha subunits have been identified and six different beta subunits have been identified. The various alpha subunits can combine with various beta subunits to form distinct integrins. Monoclonal antibodies useful in the methods and compositions of the present invention include for example HP2/1, TY21.6, TY21.12 and L25. These antibodies react with the α chain of α4β1 and block binding to VCAM-1, fibronectin and inflamed brain endothelial cells but do not affect the activity of the other members of the β1 integrin family.

Other reagents which selectively react against the VLA-4/NCAM-1 target are also envisioned. For example, an antibody which interacts with the VCAM-1 binding domain VLA-4 (α4) in conjunction with the β1 chain would block only CEP migration. Such a reagent would not affect matrix interactions (mediated by all members of the β1 integrins) nor would it affect normal intestinal immunity (mediated by integrin α4β7). The production of this and other such reagents are well within the skill of the art.

It should be recognized that for therapeutic purposes, therapeutically effective compositions for preventing CEP homing to VDA-treated tumors containing such VLA-4 or VCAM-1 directed reagents are contemplated as being within the scope of the present invention. For example, therapeutic compositions including at least one VLA-4 antagonist or VCAM-1 antagonist as well as other therapeutic compositions could be used to enhance activity of a VDA. Peptides, or peptidomimetics or other such molecules, which serve to substantially mimic one cell adhesion molecule or the other could be used in competition therapy wherein such peptides or peptidomimetics or other such molecules compete for the available locations on the surface of either the leukocyte (if substantially mimicking VCAM-1 or other VLA-4 ligand) or the endothelial cell (if substantially mimicking VLA-4).

Other art-recognized integrin α4β1 antagonists useful in the methods of the invention are disclosed, for example, in U.S. Pat. Nos. 5,730,978; 5,840,299; 6,033,665; 6,602,503; 6,960,597; 7,193,108 and 7,335,673 incorporated herein by reference. Useful integrin α4β1 antagonists are also disclosed in PCT Publications WO2006/115918, WO2006/113199, WO2006/023396, WO2005/087760, WO01/42192, WO2006/127584, WO2006/010054, WO03/011288, WO02/74761, WO02/72573, WO02/14272, WO01/14328, WO01/12128, WO00/71572, and WO98/5381. Particularly preferred for use in the present invention is an anti-integrin α4β1 antibody. Examples of antibodies are disclosed, for example, in U.S. Pat. No. 5,730,978, U.S. Pat. No. 5,840,299, U.S. Pat. No. 6,033,665, U.S. Pat. No. 6,602,503, PCT publications WO98/19790, WO2005/117976, WO91/07977, WO90/03983, WO96/08564, WO 97/18838 and published European patent application EP 0917878A1.

C. Two-Component Combination Therapy In accordance with the present invention, improved, two-component chemotherapeutic regimens comprising a VDA (e.g., a combretastatin) and an α4β1 integrin antagonist are provided for the treatment of cancer. The improved chemotherapeutic regimens can enhance efficacy for the treatment of neoplastic disease. For example, the present methods permit a clinician to administer a combretastatin compound, and an α4β1 integrin antagonist, at dosages which are significantly lower than those employed for the single agent. Preferred dosages suitable for administration of the compound of Formula I and combretastatin compounds in accordance with the invention are set forth herein below. Whether administered simultaneously or sequentially, the combretastatin compound and the at least one anticancer agent can be administered in any amount or by any route of administration effective for the modulation of tumor growth or metastasis, especially treatment of cancer as described herein.

The phrase “combination therapy” (or “co-therapy”) embraces the administration of a integrin antagonist and VDA, as part of a specific treatment regimen intended to provide a beneficial effect from the co-action of the integrin antagonist and the VDA. The beneficial effect of the combination includes, but is not limited to, pharmacokinetic or pharmacodynamic co-action resulting from the combination of the integrin antagonist and the radiation therapy. Administration of the integrin antagonist and the VDA in combination typically is carried out over a defined time period (usually minutes, hours, days or weeks depending upon the combination selected). “Combination therapy” generally is not intended to encompass the administration of an integrin antagonist and VDA as part of separate monotherapy regimens that incidentally and arbitrarily result in the combinations of the present invention. “Combination therapy” is intended to embrace administration of integrin antagonist and VDA in a sequential manner, that is, wherein the integrin antagonist and the VDA are administered at different times, as well as administration of the integrin antagonist and VDA in a substantially simultaneous manner. Substantially simultaneous administration can be accomplished, for example, by administering to the subject concurrently a single dosage having a fixed ratio of each therapeutic agent or in multiple, single dosage for each therapeutic agent. Sequential or substantially simultaneous administration of each therapeutic agent can be effected by any appropriate route including, but not limited to, oral routes, intravenous routes, intramuscular routes, and direct absorption through mucous membrane tissues. The therapeutic agents, if more than one, can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the integrin antagonist and VDA are administered is not narrowly critical although the VDA typically will follow the administration of the integrin antagonist. “Combination therapy” also can embrace the administration of the integrin antagonist and VDA as described above in further combination with other biologically active ingredients (such as, but not limited to, an antineoplastic agent) and non-drug therapies (such as, but not limited to, surgery).

In one exemplary embodiment, a combretastatin prodrug (e.g. CA4P or CA1P) is administered together with natalizumab. In a particularly preferred embodiment, a pharmaceutical composition comprising natalizumab and CA1P are used to treat cancer in a subject, wherein the subject is human. In another preferred embodiment, a pharmaceutical composition comprising natalizumab and CA4P are used to treat cancer in a subject, wherein the subject is human.

A suitable dose per day for each of the compounds, i.e., an α4β1 integrin antagonist, and a VDA (e.g. a combretastatin), can be, individually, in the range of from about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg.

Other suitable doses for the compounds of the invention include, for example, 0.1 mg/kg to about 100 mg/kg; from about 1 mg/kg to about 100 mg/kg; from about 5 mg/kg to about 50 mg/kg; from about 10 to about 25 mg/kg; about 10 mg/kg; about 15 mg/kg; about 20 mg/kg; about 25 mg/kg; about 30 mg/kg; about 40 mg/kg; about 50 mg/kg; about 60 mg/kg; about 70 mg/kg; about 80 mg/kg; about 90 mg/kg; and about 100 mg/kg. In a preferred embodiment, the VDA (e.g., a combretastatin agent) is administered at a dose ranging from between 45 mg/kg and 63 mg/kg.

D. Pharmaceutical Compositions

As explained above, the present methods can, for example, be carried out using a single pharmaceutical composition comprising both a VDA and an α4β1 integrin antagonist when administration is to be simultaneous or sequential.

Pharmaceutical compositions employed in the methods of the invention include a compound (e.g., a VDA and/or α4β1 integrin antagonist) formulated with other ingredients, e.g., “pharmaceutically acceptable carriers”. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers, for example to a diluent, adjuvant, excipient, auxiliary agent or vehicle with which an active agent of the present invention is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Other pharmaceutical carriers include, but are not limited to, antioxidants, preservatives, dyes, tablet-coating compositions, plasticizers, inert carriers, excipients, polymers, coating materials, osmotic barriers, devices and agents which slow or retard solubility, etc. Non-toxic pharmaceutically acceptable excipients that are suitable for the manufacture of tablets include, for example, inert diluents, such as calcium carbonate, sodium carbonate, lactose, calcium phosphate or sodium phosphate; granulating and disintegrating agents, for example, corn starch, or alginic acid; and binding agents, for example magnesium stearate, stearic acid or talc. Suitable pharmaceutical carriers are described in “Remington's Pharmaceutical Sciences” by E. W. Martin.

A pharmaceutical composition of the present invention can be administered by any suitable route, for example, by injection, by oral, pulmonary, nasal or other forms of administration. In general, pharmaceutical compositions contemplated to be within the scope of the invention, comprise, inter alia, pharmaceutically acceptable diluents, preservatives, solubilizers, emulsifiers, adjuvants and/or carriers. Such compositions can include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc., or into liposomes. Such compositions may influence the physical state, stability, rate of in vivo release, and rate of in vivo clearance of components of a pharmaceutical composition of the present invention. See, e.g., Remington's Pharmaceutical Sciences, 18th Ed. (1990, Mack Publishing Co., Easton, Pa. 18042) pages 1435-1712 which are herein incorporated by reference. A pharmaceutical composition of the present invention can be prepared, for example, in liquid form, or can be in dried powder, such as lyophilized form. Particular methods of administering such compositions are described infra.

Suitable pharmaceutical compositions for oral use, include, but are not limited to, tablets, troches, lozenges, aqueous or oily suspensions, dispersible powders or granules, emulsions, hard or soft capsules, solutions, syrups and elixirs. Compositions intended for oral use may be prepared according to any method known to the art for the manufacture of pharmaceutical compositions. Such compositions may contain one or more agents selected from the group consisting of diluents, sweetening agents, flavoring agents, coloring agents and preserving agents in order to provide palatable preparations. Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions containing the active materials in admixture with excipients suitable for the manufacture of aqueous suspensions may also be used. Such excipients are suspending agents, for example sodium carboxymethylcellulose, methylcellulose, hydroxypropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethylene oxycetanol, or condensation products of ethylene oxide with partial esters derived from fatty acids and hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl hydroxybenzoate, one or more coloring agents, one or more flavoring agents, and one or more sweetening agents, such as sucrose or saccharin.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example, sweetening, flavoring and coloring agents, may also be present.

The compounds of the invention may also be in the form of non-aqueous liquid formulations, e.g., oily suspensions which may be formulated by suspending the active ingredients in a vegetable oil, for example arachis oil, olive oil, sesame oil or peanut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The compounds of the invention may also be administered in the form of suppositories for rectal or vaginal administration of the drug. These compositions can be prepared by mixing the drug with a suitable non-irritating excipient which is solid at ordinary temperatures but liquid at the rectal temperature or vaginal temperature and will therefore melt in the rectum or vagina to release active drug.

Diseases which can be treated in accordance with present invention include, but are not limited: Accelerated Phase Chronic Myelogenous Leukemia; Acute Erythroid Leukemia; Acute Lymphoblastic Leukemia; Acute Lymphoblastic Leukemia in Remission; Acute Lymphocytic Leukemia; Acute Monoblastic and Acute Monocytic Leukemia; Acute Myelogenous Leukemia; Acute Myeloid Leukemia; Adenocarcinoma; Adenocarcinoma of the Colon; Adenocarcinoma of the Esophagus; Adenocarcinoma of the Lung; Adenocarcinoma of the Pancreas; Adenocarcinoma of the Prostate; Adenocarcinoma of the Rectum; Adenocarcinoma of the Stomach; Adenoid Cystic Carcinoma of the Head and Neck; Adenosquamous Cell Lung Cancer; Adult Giant Cell Glioblastoma; Advanced Adult Primary Liver Cancer; Advanced Gastrointestinal Stromal Tumor; Advanced Non-Nasopharyngeal Head and Neck Carcinoma; Advanced NSCLC; Advanced Solid Tumors; Agnogenic Myeloid; Metaplasia; Anaplastic Astrocytoma; Anaplastic Oligodendroglioma; Anaplastic Thyroid Cancer; Astrocytoma; Atypical Chronic Myelogenous Leukemia; B-Cell Adult Acute Lymphoblastic Leukemia; Bladder Cancer; Blastic Phase Chronic Myelogenous Leukemia; Bone Metastases; Brain Tumor; Breast Cancer; Breast Cancer in Situ; Breast Neoplasms; Brenner Tumor; Bronchoalveolar Cell Lung Cancer; Cancer of the Fallopian Tube; Carcinoma, Squamous Cell; Central Nervous System Cancer; Cervix Neoplasms; Childhood Acute Lymphoblastic Leukemia; Childhood Acute Lymphoblastic Leukemia in Remission; Childhood Brain Tumor; Childhood Central Nervous System Germ Cell Tumor; Childhood Cerebellar Astrocytoma; Childhood Chronic Myelogenous Leukemia; Childhood Ependymoma; Childhood Malignat Germ Cell Tumor; Childhood Oligodendroglioma; Childhood Soft Tissue Sarcoma; Chordoma; Chronic Eosinophilic Leukemia (CEL); Chronic Idiopathic Myelofibrosis; Chronic Myelogenous Leukemia; Chronic Myeloid Leukemia; Chronic Myelomonocytic Leukemia; Chronic Phase Chronic Myelogenous Leukemia; Colon Cancer; Colorectal Cancer; Congenital Fibrosarcoma; Dermatofibrosarcoma; Dermatoftbrosarcoma Protuberans (DFSP); Desmoid Tumor; Endometrial Adenocarcinoma; Endometrial Adenosquamous Cell; Eosinophilia; Esophageal Cancer; Epidemic Kaposi's Sarcoma; Epithelial Mesothelioma; Esophageal Cancer; Esophagogastric Cancer; Essential Thrombocythemia; Ewing's Family of Tumors; Extensive Stage Small Cell Lung Cancer; Extrahepatic Bile Duct Cancer; Fallopian Tube Cancer; Familiar Hypereosinophilia; Fibrosarcoma; Follicular Thyroid Cancer; Gallbladder Cancer; Gastric Adenocarcinoma; Gastric Cancer; Gastroinstestinal Cancer; Gastrinoma; Gastrointestinal Carcinoid; Gastrointestinal Neoplasm; Gastrointestinal Stromal Tumor; Giant Cell Glioblastoma; Glioblastoma; Glioma; Glioblastoma Multiforme; Gliosarcoma; Grade I Meningioma; Grade II Meningioma; Grade III Meningioma; Head and Neck Cancer; Head and Neck Neoplasms; Hematopoietic and Lymphoid Cancer, Hepatocellular Carcinoma; High-Grade Childhood Cerebral Astrocytoma; Hypereosinophilic Syndrome; Hypopharyngeal Cancer; Idiopathic Pulmonary Fibrosis; Inflammatory Myofibroblastic Tumor; Inoperable Locally Advanced Squamous Cell Carcinoma of Head and Neck; Insulinoma; Intraductal Breast Carcinoma; Islet Cell Carcinoma; Kidney and Urinary Cancer; L1 Adult Acute Lymphoblastic Leukemia; L2 Adult Acute Lymphoblastic Leukemia; Large Cell Lung Cancer; Laryngeal Cancer; Leukemia, Lymphocytic, Acute L2; Leukemia, Myeloid, Chronic; Leukemia, Myeloid, Chronic Phase; Lip and Oral Cavity Cancer; Lip Cancer; Liver Cancer; Liver Dysfunction and Neoplasm; Localized Unresectable Adult Primary Liver Cancer; Low-Grade Childhood Cerebral Astrocytoma; Lymphoid Blastic Phase of Chronic Myeloid Leukemia; Lung Adenocarcinoma With Bronchiole-Alveolar Feature; Lung Cancer; Male Breast Cancer; Malignant Fibrous Histiocytoma; Malignant Melanoma; Mastocytosis; Medullary Thyroid Cancer; Melanoma; Meningeal Tumors; Meningeal Hemangiopericytoma; Meningioma; Meningioma; Meningioma; Mesothelioma; Metastatic Cancer; Metastatic Solid Tumors; Metastatic Colorectal Cancer; Metastatic Gastrointestinal Carcinoid Tumor; Metastatic Pancreatic Carcinoma; Mixed Gliomas; Multiple Myeloma; Musculoskeletal Tumors; Myelodysplastic Syndrome; Myelogenous Leukemia, Acute; Myelofibrosis; Myeloid Leukemia, Chronic; Myeloid Leukemia, Chronic Accelerated-Phase; Myeloid Leukemia, Chronic, Chronic-Phase; Myeloid Metaplasia; Myeloproliferative Disorder (MPD) with Eosinophilia; Nasopharyngeal Cancer; Nasopharyngeal Carcinoma; Neoplasms; Neuroblastoma; Neurofibrosarcoma; Non-B Childhood Acute Lymphoblastic Leukemia; Non-Metastatic (T2-T4, N0-N3, MO; Stages II and III) and Histologically-Confirmed Intestinal GC; Non-Metastatic Prostate Cancer; Nonresectable Adrenocortical Carcinoma; Non-Small Cell Lung Cancer; Nose Cancer; Oligodendroglioma; Oligodendroglial Tumors; Oral Cancer; Oropharyngeal Cancer; Osteosarcoma; Ovarian Cancer; Ovarian Epithelial Cancer; Ovarian Germ Cell Tumor; Ovarian Low Malignant Potential Tumor; Ovarian Neoplasms; Pancreatic Cancer; Papillary Thyroid Cancer; Pelvic Neoplasms; Peritoneal Cavity Cancer; Peritoneal Carcinoma; Peritoneal Neoplasms; Pharynx Cancer; Philadelphia Chromosome Positive Chronic Myelogenous Leukemia; Philadelphia Positive Acute Lymphoblastic Leukemia; Philadelphia Positive Chronic Myeloid Leukemia in Myeloid Blast Crisis; Pneumonic-Type Adenocarcinoma (P-ADC); Polycythemia Vera; Pulmonary Fibrosis; Primary Hepatocellular Carcinoma; Primary Liver Cancer; Prostate Cancer; Prostate Cancer, Antigen Independent; Rectal Cancer; Recurrent Adult Brain Tumor; Recurrent Adult Soft Tissue Sarcoma; Recurrent Adult Primary Liver Cancer; Recurrent Breast Cancer; Recurrent Cervical Cancer; Recurrent Colon Cancer; Recurrent Endometrial Cancer, Recurrent Esophageal Cancer; Recurrent Gastric Cancer; Recurrent Glioblastoma; Recurrent Glioblastoma Multiforme (GBM); Recurrent Kaposi's Sarcoma; Recurrent Melanoma; Recurrent Merkel Cell Carcinoma; Recurrent Ovarian Epithelial Cancer; Recurrent Pancreatic Cancer; Recurrent Prostate Cancer; Recurrent Rectal Cancer; Recurrent Salivary Gland Cancer; Recurrent Skin Cancer; Recurrent Small Cell Lung Cancer; Recurrent Tumors of the Ewing's Family; Recurrent Uterine Sarcoma; Refractory Germ Cell Tumors Expressing EGRF; Relapsing Chronic Myelogenous Leukemia; Renal Cell Cancer; Renal Cell Carcinoma; Renal Papillary Carcinoma; Rhabdomyosarcomas; Salivary Gland Adenoid Cystic Carcinoma; Sarcoma; Sarcomatous Mesothelioma; Skin Cancer; Small Cell Lung Cancer; Soft Tissue Sarcoma; Squamous Cell Carcinoma; Squamous Cell Carcinoma of the Esophagus; Squamous Cell Carcinoma of the Head and Neck; Squamous Cell Carcinoma of the Skin; Squamous Cell Lung Cancer; Stage II Esophageal Cancer; Stage III Esophageal Cancer, Stage II Melanoma; Stage II Merkel Cell Carcinoma; Stage III Adult Soft Tissue Sarcoma; Stage III Esophageal Cancer; Stage III Merkel Cell Carcinoma; Stage III Ovarian Epithelial Cancer; Stage III Pancreatic Cancer; Stage III Salivary Gland Cancer; Stage III B Breast Cancer; Stage III C Breast Cancer; Stage IV Adult Soft Tissue Sarcoma; Stage IV Breast Cancer; Stage IV Colon Cancer; Stage IV Esophageal Cancer; Stage IV Gastric Cancer; Stage IV Melanoma; Stage IV Ovarian Epithelial Cancer; Stage IV Prostate Cancer; Stage IV Rectal Cancer; Stage IV Salivary Gland Cancer; Stage IVA Pancreatic Cancer; Stage IVB Pancreatic Cancer; Systemic Mastocytosis; Synovial Sarcoma; T-lymphoma; T-Cell Childhood Acute Lymphoblastic Leukemia; Testicular Cancer; Thorax and Respiratory Cancer; Throat Cancer; Thyroid Cancer; Transitional Cell Cancer of the Renal Pelvis and Ureter; Transitional Cell Carcinoma of the Bladder; Tubal Carcinoma; Unresectable or Metastatic Malignant Gastrointestinal Stromal Tumor (GIST); Unspecified Childhood Solid Tumor; Unspecified Adult Solid Tumor; Untreated Childhood Brain Stem Glioma; Urethral Cancer; Uterine Carcinosarcoma, and Uterine Sarcoma.

As explained above, the present invention is directed towards methods for modulating tumor growth and metastasis comprising, inter alia, the administration of a VDA and an α4β1 integrin antagonist. The agents of the invention can be administered separately (e.g., formulated and administered separately), or in combination as a pharmaceutical composition of the present invention. Administration can be achieved by any suitable route, such as parenterally, transmucosally, e.g., orally, nasally, or rectally, or transdermally. Preferably, administration is parenteral, e.g., via intravenous injection. Alternative means of administration also include, but are not limited to, intra-arteriole, intramuscular, intradermal, subcutaneous, intraperitoneal, intraventricular, and intracranial administration/or by injection into the tumor(s) being treated or into tissues surrounding the tumor(s).

The pharmaceutical composition may be employed in any suitable pharmaceutical formulation, as described above, including in a vesicle, such as a liposome [see Langer, Science 249:1527-1533 (1990); Treat et al. in Liposomes in the Therapy of Infectious Disease and Cancer, Lopez-Berestein and Fidler (eds.), Liss: New York, pp. 317-327, see generally, ibid] Preferably, administration of liposomes containing the agents of the invention is parenteral, e.g., via intravenous injection, but also may include, without limitation, intra-arteriole, intramuscular, intradermal, subcutaneous, intraperitoneal, intraventricular, and intracranial administration, or by injection into the tumor(s) being treated or into tissues surrounding the tumor(s).

In yet another embodiment, a pharmaceutical composition of the present invention can be delivered in a controlled release system, such as using an intravenous infusion, an implantable osmotic pump, a transdermal patch, liposomes, or other modes of administration. In a particular embodiment, a pump may be used [see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)]. In another embodiment, polymeric materials can be used [see Medical Applications of Controlled Release, Langer and Wise (eds.)/CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)]. In yet another embodiment, a controlled release system can be placed in proximity of the target tissues of the animal, thus requiring only a fraction of the systemic dose [see, e.g., Goodson, in Medical Applications of Controlled Release, supra, vol. 2, pp. 115-138 (1984).]. In particular, a controlled release device can be introduced into an animal in proximity of the site of inappropriate immune activation or a tumor. Other controlled release systems are discussed in the review by Langer [Science 249:1527-1533 (1990)].

A controlled release formulation can be pulsed, delayed, extended, slow, steady, immediate, rapid, fast, etc. It can comprise one or more release formulations, e.g. extended- and immediate-release components. Extended delivery systems can be utilized to achieve a dosing internal of once every 24 hours, once every 12 hours, once every 8 hours, once every 6 hours, etc. The dosage form/delivery system can be a tablet or a capsule suited for extended release, but a sustained release liquid or suspension can also be used. A controlled release pharmaceutical formulation can be produced which maintains the release of, and or peak blood plasma levels of a compound of the invention.

Compounds of the invention may also be administrated transdermally using methods known to those skilled in the art (see, for example: Chien; “Transdermal Controlled Systemic Medications”; Marcel Dekker, Inc.; 1987. Lipp et al. WO94/04157 3 Mar. 1994). For example, a solution or suspension of a compound of the invention in a suitable volatile solvent optionally containing penetration enhancing agents can be combined with additional additives known to those skilled in the art, such as matrix materials and bactericides. After sterilization, the resulting mixture can be formulated following known procedures into dosage forms. In addition, on treatment with emulsifying agents and water, a solution or suspension of a compound of the invention may be formulated into a lotion or salve.

Suitable solvents for processing transdermal delivery systems are known to those skilled in the art, and include lower alcohols such as ethanol or isopropyl alcohol, lower ketones such as acetone, lower carboxylic acid esters such as ethyl acetate, polar ethers such as tetrahydrofuran, lower hydrocarbons such as hexane, cyclohexane or benzene, or halogenated hydrocarbons such as dichloromethane, chloroform, trichlorotrifluoroethane, or trichlorofluoroethane. Suitable solvents may also include mixtures of one or more materials selected from lower alcohols, lower ketones, lower carboxylic acid esters, polar ethers, lower hydrocarbons, halogenated hydrocarbons.

Suitable penetration enhancing materials for transdermal delivery system are known to those skilled in the art, and include, for example, monohydroxy or polyhydroxy alcohols such as ethanol, propylene glycol or benzyl alcohol, saturated or unsaturated C8-C18 fatty alcohols such as lauryl alcohol or cetyl alcohol, saturated or unsaturated C8-C18 fatty acids such as stearic acid, saturated or unsaturated fatty esters with up to 24 carbons such as methyl, ethyl, propyl, isopropyl, n-butyl, sec-butyl, isobutyl, tertbutyl or monoglycerin esters of acetic acid, capronic acid, lauric acid, myristinic acid, stearic acid, or palmitic acid, or diesters of saturated or unsaturated dicarboxylic acids with a total of up to 24 carbons such as diisopropyl adipate, diisobutyl adipate, diisopropyl sebacate, diisopropyl maleate, or diisopropyl fumarate. Additional penetration enhancing materials include phosphatidyl derivatives such as lecithin or cephalin, terpenes, amides, ketones, ureas and their derivatives, and ethers such as dimethyl isosorbid and diethyleneglycol monoethyl ether. Suitable penetration enhancing formulations may also include mixtures of one or more materials selected from monohydroxy or polyhydroxy alcohols, saturated or unsaturated C 8-C 18 fatty alcohols, saturated or unsaturated 08-C18 fatty acids, saturated or unsaturated fatty esters with up to 24 carbons, diesters of saturated or unsaturated discarboxylic acids with a total of up to 24 carbons, phosphatidyl derivatives, terpenes, amides, ketones, ureas and their derivatives, and ethers.

Suitable binding materials for transdermal delivery systems are known to those skilled in the art and include polyacrylates, silicones, polyurethanes, block polymers, styrenebutadiene copolymers, and natural and synthetic rubbers. Cellulose ethers, derivatized polyethylenes, and silicates may also be used as matrix components. Additional additives, such as viscous resins or oils may be added to increase the viscosity of the matrix.

E. Synthetic Procedure

Compounds of the present invention are prepared from commonly available compounds using procedures known to those skilled in the art, including any one or more of the following conditions without limitation:

Within the scope of this text, only a readily removable group that is not a constituent of the particular desired end product of the compounds of the present invention is designated a “protecting group,” unless the context indicates otherwise. The protection of functional groups by such protecting groups, the protecting groups themselves, and their cleavage reactions are described for example in standard reference works, such as e.g., Science of Synthesis: Houben-Weyl Methods of Molecular Transformation. Georg Thieme Verlag, Stuttgart, Germany. 2005. 41627 pp. (URL: http://www.science-of-synthesis.com (Electronic Version, 48 Volumes)); J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide and Derivate” (Chemistry of Carbohydrates: Monosaccharides and Derivatives), Georg Thieme Verlag, Stuttgart 1974. A characteristic of protecting groups is that they can be removed readily (i.e., without the occurrence of undesired secondary reactions) for example by solvolysis, reduction, photolysis or alternatively under physiological conditions (e.g., by enzymatic cleavage).

Acid addition salts of the compounds of the invention are most suitably formed from pharmaceutically acceptable acids, and include for example those formed with inorganic acids e.g. hydrochloric, hydrobromic, sulphuric or phosphoric acids and organic acids e.g. succinic, maleic, acetic or fumaric acid. Other non-pharmaceutically acceptable salts e.g. oxalates can be used for example in the isolation of the compounds of the invention, for laboratory use, or for subsequent conversion to a pharmaceutically acceptable acid addition salt. Also included within the scope of the invention are solvates and hydrates of the invention.

The conversion of a given compound salt to a desired compound salt is achieved by applying standard techniques, in which an aqueous solution of the given salt is treated with a solution of base e.g. sodium carbonate or potassium hydroxide, to liberate the free base which is then extracted into an appropriate solvent, such as ether. The free base is then separated from the aqueous portion, dried, and treated with the requisite acid to give the desired salt.

In vivo hydrolyzable esters or amides of certain compounds of the invention can be formed by treating those compounds having a free hydroxy or amino functionality with the acid chloride of the desired ester in the presence of a base in an inert solvent such as methylene chloride or chloroform. Suitable bases include triethylamine or pyridine. Conversely, compounds of the invention having a free carboxy group can be esterified using standard conditions which can include activation followed by treatment with the desired alcohol in the presence of a suitable base.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzenesulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Particularly preferred salts are sodium, lysine and arginine salts of the compounds of the invention. Such salts can be formed by procedures well known and described in the art.

Other acids such as oxalic acid, which can not be considered pharmaceutically acceptable, can be useful in the preparation of salts useful as intermediates in obtaining a chemical compound of the invention and its pharmaceutically acceptable acid addition salt.

Metal salts of a chemical compound of the invention include alkali metal salts, such as the sodium salt of a chemical compound of the invention containing a carboxy group.

Mixtures of isomers obtainable according to the invention can be separated in a manner known per se into the individual isomers; diastereoisomers can be separated, for example, by partitioning between polyphasic solvent mixtures, recrystallisation and/or chromatographic separation, for example over silica gel or by, e.g., medium pressure liquid chromatography over a reversed phase column, and racemates can be separated, for example, by the formation of salts with optically pure salt-forming reagents and separation of the mixture of diastereoisomers so obtainable, for example by means of fractional crystallisation, or by chromatography over optically active column materials.

Intermediates and final products can be worked up and/or purified according to standard methods, e.g., using chromatographic methods, distribution methods, (re-) crystallization, and the like.

Those skilled in the art will recognize, or be able to ascertain using no more than routine experimentation, many equivalents to the specific embodiments of the invention described herein. Such equivalents are intended to be encompassed by the following claims.

The following examples are provided to illustrate embodiments of the invention. They are not intended to limit the invention in any way.

### V. Example

Integrin ∘4β1 (VLA4) promotes the homing of circulating cells to the α4β1 ligand binding (VCAM) expressed at the tumor site (Jin et al. 2006. J Clin Invest 116:652-662). They also showed that bone marrow CD34⁺ progenitor cells, which express integrin α4β1, home to sites of active tumor neovascularization, and that this can be blocked by a treatment with an α4β1 blocking antibody (Jin 2006). We determined whether α4β1 integrin contributes to CEP and possibly other types of bone-marrow pro-angiogenic cell invasion and homing to the viable tumor rim after VDA treatment. To this end, green fluorescent protein (GFP)positive bone marrow cells obtained from syngeneic UBI/GFP/BL6 donor mice (The Jackson Laboratory, Bar Harbor, Me.) were transplanted into lethally irradiated C57Bl/6 recipient mice (as described in Theise, et al. 2000. Hepatology 31:235-240). Three weeks later, mice were subcutaneously (s.c.) implanted with 0.5×10⁶ Lewis Lung Carcinoma (LLC) cells (ATCC, Manassas, Va.).

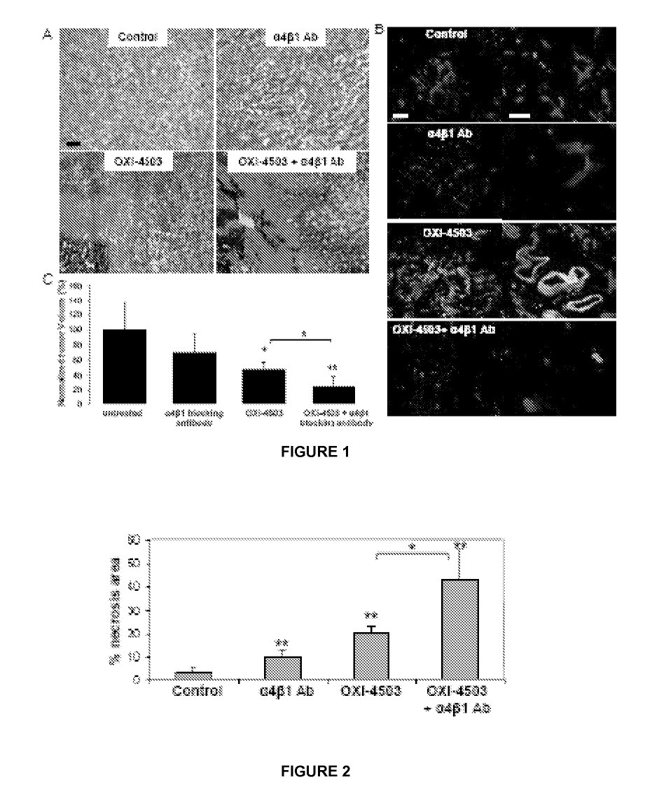

When tumors reached 500 mm³, mice were treated with an integrin α4β1 specific blocking antibody (Jin 2006), CA1P, or a combination of the two agents. After 3 days, tumor volumes were measured, mice were sacrificed and tumors were removed for the evaluation of necrosis (FIG. 1A) and incorporation of bone marrow cells to the tumor vasculature (FIG. 1B). Analysis of tumor necrosis was carried out by calculating the fraction of the tumor area demonstrating tumor tissue autofluorescence for necrosis (green). FIG. 1C provides tumor volumes measured three days after treatment and normalized to untreated control tumors. *: 0.05>p>0.01; **: p<0.01. Tumor size was assessed with Vernier calipers by using the formula width²×length×0.5.

Tumor sections were visualized under a Carl Zeiss Axioplan 2 microscope (Carl Zeiss Canada Inc. Toronto, ON, Canada), using bright field and fluorescence filters: GFP (470 nm excitation) for GFP bone marrow cell staining or autofluorescence of tissue necrosis. Images were captured with a Zeiss Axiocam digital camera connected to the microscope using AxioVision 3.0 software. The number of fields per tumor sample varied from 5 to 20, depending on the tumor size and microscope magnification. Magnification of 25× was used for the analysis of necrosis in the entire tumor section. To quantify necrotic fractions, as well as vascular space and cellularity in bones, Adobe Photoshop 6.0 software (Adobe systems incorporated, San Jose, Calif.) was used and the percentage of necrotic or vascular space areas as well as cellular density was calculated from the total tumor/bone area. In all cases, a total of at least 20 fields per group were analyzed. The cross-sections were made along the longest tumor/bone diameter to allow all the tumor/bone areas to be represented in the sample. The 15-20 μm cyrosections from LLC tumors in GFP+ bone marrow transplanted mice were analyzed on a Zeiss Axiovert 100 M confocal microscope.

In FIG. 2, necrosis in LLC tumors from FIG. 1A (n>20 fields/group) were quantified and plotted as the percentage of green (necrosis) pixels from total pixel area (*: 0.05>p>0.01; **: p<0.01). Tumors treated with a combination of α4β1 blocking antibody and CA1P were significantly smaller, and exhibited a significant increase in tumor necrosis, with a less prominent viable tumor rim, compared to CA1P treatment alone. No significant difference in tumor necrosis between α4β1 blocking antibody treated and control untreated tumors was observed.

Next, in order to evaluate whether α4β1 blocking antibody treatment inhibits the homing and invasion of CEPs to the viable tumor rim, sections from harvested tumors were stained for CD31 (in red) and assessed by confocal microscopy. Blood vessels were immunostained with an anti-CD31 antibody (1:200, BD Pharmingen, San Diego, Calif.) and its secondary Cy3-conjugated donkey anti-rat antibody (1:200, Jackson ImmunoResearch Laboratories Inc., West Grove, Pa.). Tumor sections were visualized under a Carl Zeiss Axioplan 2 microscope (Carl Zeiss Canada Inc. Toronto, ON, Canada), using bright field and fluorescence filters: Cy3 (540 nm excitation) for CD31 staining; and GFP (470 nm excitation) for GFP bone marrow cell staining. Images were captured with a Zeiss Axiocam digital camera connected to the microscope using AxioVision 3.0 software. The results in FIG. 1B show that CA1P treated tumors revealed a massive invasion of GFP⁺ cells (bone marrow cells designated in green) in the viable tumor rim, some of which were found to incorporate into blood vessels. However, α4β1 blocking antibody treatment inhibited the invasion/homing of GFP⁺ cells of the viable tumor rim, with minimal evidence for colocalization of GFP⁺ and CD31⁺ cells.

The addition of α4β1 neutralizing antibodies can block tumor homing/retention of such cells and hence increase the efficacy of VDA treatment.

1. A method for producing an anti-tumor effect in a subject suffering from cancer or a tumor, the method comprising administering to the patient a Vascular Disrupting Agent (VDA) and an α4β1 integrin antagonist in amounts effective therefor.

2. (canceled)

3. (canceled)

4. A method for inhibiting homing and retention of circulating endothelial progenitor (CEP) cells or other proangiogenic cells to the tumor of a subject that is treated with a VDA, the method comprising administering to the patient an α4β1 integrin antagonist in amounts effective therefor.

5. The method of claim 1, wherein the α4β1 integrin antagonist is an antibody.

6. The method of claim 1, wherein the VDA is a combretastatin agent.

7. The method of claim 1, wherein the combretastatin agent is a compound of Formula II: 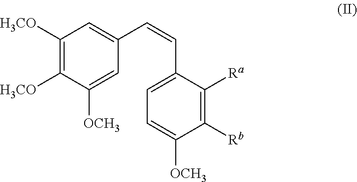
or a pharmaceutically acceptable salt thereof, wherein
R^{a} is H, phosphate, phosphate ester, phosphonate, phosphoramidate monoester, phosphoramidate diester, cyclic phosphoramidate, phosphordiamidate, cyclic phosphorodiamidate, phosphonamidate or amino acid acyl; and
R^{b} is phosphate, phosphate ester, phosphonate, phosphoramidate monoester, phosphoramidate diester, cyclic phosphoramidate, phosphordiamidate, cyclic phosphorodiamidate, phosphonamidate or amino acid acyl.

8. The method of claim 1, wherein the combretastatin agent is a compound of Formula IIb: 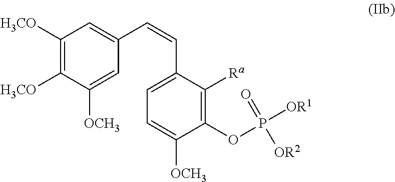
wherein
R^{a} is H or OP(O)(OR³)OR⁴; and
OR¹, OR², OR³ and OR⁴ are each, independently, H, —O-QH⁺ or —O— M⁺, wherein M⁺ is a monovalent or divalent metal cation, and Q is, independently:
a) an amino acid containing at least two nitrogen atoms where one of the nitrogen atoms, together with a proton, forms a quaternary ammonium cation QH⁺; or
b) an organic amine containing at least one nitrogen atom which, together with a proton, forms a quaternary ammonium cation, QH⁺.

9. The method of claim 7 wherein the compound of Formula II or IIb is administered at a dose ranging from between 45 mg/kg and 63 mg/kg.

10. The method of claim 8, wherein, for Formula IIb, R³ is H or OP(O)(OR³)OR⁴, and R¹, R², R³ and R⁴ are each, independently, an aliphatic organic amine, alkali metals, transition metal, heteroarylene, heterocyclyl, nucleoside, nucleotide, alkaloid, amino sugar, amino nitrile, or nitrogenous antibiotic.

11. The method of claim 8, wherein, for Formula IIb, R¹, R², R³ and R⁴ are each, independently, Na, TRIS, histidine, ethanolamine, diethanolamine, ethylenediamine, diethylamine, triethanolamine, glucamine, N-methylglucamine, ethylenediamine, 2-(4-imidazolyl)-ethylamine, choline, or hydrabamine.

12. The method of claim 7, wherein Formula II or Formula IIb is represented by a compound of Formula III: 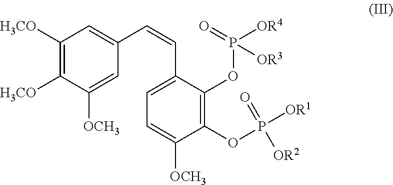
and pharmaceutically acceptable salts thereof.

13. The method of claim 1, wherein the Vascular Disrupting Agent (VDA) and α4β1 integrin antagonist are simultaneously or sequentially administered.

14. The method of claim 1, wherein said cancer is selected from the group consisting of ovarian cancer, fallopian tube cancer, cervical cancer, breast cancer, lung cancer, melanoma, and primary cancer of the peritoneum.

15. The method of claim 14, wherein said tumor is a solid tumor selected from the group consisting of a melanoma, an ovarian tumor, a cervical tumor, a breast tumor, small cell lung tumor, a non-small cell lung tumor, a fallopian tube tumor, and a primary tumor of the peritoneum.

16. (canceled)

17. (canceled)

18. (canceled)

19. A method of treating a tumor in a subject in need thereof by administering to the subject a pharmaceutical composition comprising natalizumab and CA1P or CA4P.

20. (canceled)

21. (canceled)

22. A pharmaceutical composition for producing an anti-tumor effect in a subject suffering from cancer or a tumor, comprising a Vascular Disrupting Agent (VDA) and α4β1 integrin antagonist in amounts effective therefore in a pharmaceutical carrier.

23. The composition of claim 22, wherein the α4β1 integrin antagonist is an antibody.

24. The composition of claim 22, wherein the VDA is a combretastatin agent.

25. The composition of claim 22, wherein the combretastatin agent is a compound of Formula II: 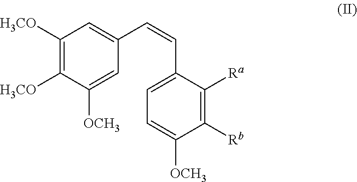
or a pharmaceutically acceptable salt thereof, wherein
R^{a} is H, phosphate, phosphate ester, phosphonate, phosphoramidate monoester, phosphoramidate diester, cyclic phosphoramidate, phosphordiamidate, cyclic phosphorodiamidate, phosphonamidate or amino acid acyl; and
R^{b} is phosphate, phosphate ester, phosphonate, phosphoramidate monoester, phosphoramidate diester, cyclic phosphoramidate, phosphordiamidate, cyclic phosphorodiamidate, phosphonamidate or amino acid acyl.

26. The composition of claim 22, wherein the combretastatin agent is a compound of Formula IIb: 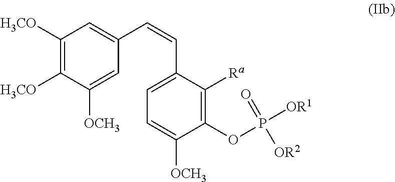
wherein
R^{a} is H or OP(O)(OR³)OR⁴; and
OR¹, OR², OR³ and OR⁴ are each, independently, H, —O-QH⁺ or —O— M⁺, wherein M⁺ is a monovalent or divalent metal cation, and Q is, independently:
a) an amino acid containing at least two nitrogen atoms where one of the nitrogen atoms, together with a proton, forms a quaternary ammonium cation QH⁺; or
b) an organic amine containing at least one nitrogen atom which, together with a proton, forms a quaternary ammonium cation, QH⁺.

27. The composition of claim 25, wherein the compound of Formula II or IIb is administered at a dose ranging from between 45 mg/kg and 63 mg/kg.

28. The composition of claim 26, wherein, for Formula IIb, R³ is H or OP(O)(OR³)OR⁴, and R¹, R², R³ and R⁴ are each, independently, an aliphatic organic amine, alkali metals, transition metal, heteroarylene, heterocyclyl, nucleoside, nucleotide, alkaloid, amino sugar, amino nitrile, or nitrogenous antibiotic.

29. The composition of claim 26, wherein, for Formula IIb, R¹, R², R³ and R⁴ are each, independently, Na, TRIS, histidine, ethanolamine, diethanolamine, ethylenediamine, diethylamine, triethanolamine, glucamine, N-methylglucamine, ethylenediamine, 2-(4-imidazolyl)-ethylamine, choline, or hydrabamine.

30. The composition of claim 22, wherein Formula II or IIb is represented by a compound of Formula III: 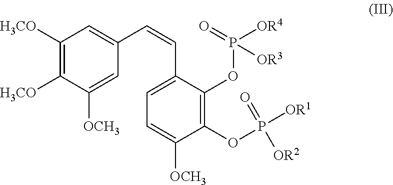
and pharmaceutically acceptable salts thereof.

31. The composition of claim 22, said pharmaceutical composition comprising natalizumab and CA1P or CA4P.

32. (canceled)
